SEQUENCE LISTING (3 sequences)

SEQ ID NO: 1 - protein, 166 aa, Homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 2 - protein, 166 aa, Artificial Sequence
  HYBRID 1
CDLPQTHSLGNRRALILLGQMGRISPFSCLKDRHDFRIPQEEFDGNQFQKAQAISVLHEMMQQTFNLFSTENSSAAWEQTLLEKFSIELFQQMNDLEACVIQEVGVEETPLMNEDSILAVRKYFQRITLYLIERKYSPCAWEVVRAEIMRSLSFSTNLQKRLRRKD

SEQ ID NO: 3 - protein, 166 aa, Artificial Sequence
  HYBRID with 11 Mutations
CDLPQTHSLGNRRALILLGQMGRISPFSCLKDRHDFRIPQEEFDGNQFQKAQAISVLHEMMQQTFNLFSTKNSSAAWDETLLEKFYIELFQQMNDLEACVIQEVGVEETPLMNEDSILAVKKYFQRITLYLIERKYSPCAWEVVRAEIMRSLSFSTNLQKRLRRKD

(19) United States
(12) Patent Application Publication
(10) Pub. No.: US 2021/0228687 A1
(43) Pub. Date: Jul. 29, 2021

(54) COMPOSITIONS AND METHODS RELATING TO THE TREATMENT OF DISEASES

(71) Applicant: ILC Therapeutics LTD, Lanarkshire (GB)

(72) Inventors: William Stimson, Newhouse (GB); Christopher McKenzie, Newhouse (GB)

(21) Appl. No.: 17/050,988

(22) PCT Filed: Jun. 3, 2019
(86) PCT No.: PCT/GB2019/051533

§ 371 Date: Oct. 27, 2020

(30) Foreign Application Priority Data
Jun. 1, 2018 (GB) 1809005.0
Mar. 15, 2019 (GB) 1903608.6

(51) Int. Cl. A61K 38/21; A61P 17/06; A61K 9/00

(52) U.S. Cl. CPC A61K 38/212; A61K 9/006; A61P 17/06

(57) ABSTRACT

There is provided a method and compositions for the treatment and/or prophylaxis of psoriasis or atopic dermatitis, the method comprising the step of administering to a subject in need thereof a therapeutically effective amount of an interferon alpha subtype, wherein the interferon 3 alpha subtype is IFN-α14, HYBRID 1 or a combination of IFN-α14 and HYBRID 1.

### FIELD OF THE INVENTION

The present invention relates to compositions and methods for preventing or treating psoriasis and/or Atopic Dermatitis, and conditions where an exaggerated Th-17 response plays a detrimental role, such as inflammatory responses and autoimmune diseases. The invention further extends to compositions and the use of the compositions of the invention for the treatment and/or prophylaxis of Psoriasis and Atopic Dermatitis for Human and Veterinary therapies.

### BACKGROUND TO THE INVENTION

An over-reactive Th1 response can generate organ-specific autoimmune disease such as arthritis, multiple sclerosis, or Type I diabetes, while an over-reactive Th2/Th17 response may underlie allergy and atrophy. It is currently believed that Th17 cells play a major role in host defence against pathogens and an exaggerated Th17 response may lead to severe inflammatory responses and autoimmune diseases, such as psoriasis. Th17 cells produce interleukin-17 (IL17). There are six known isoforms of IL17, from A to F. Both IL17A and IL17F are pro-inflammatory cytokines.

It is known that different pathogens induce different IFN-α subtypes in vitro and that IFN-α subtypes have different antiviral, anti-proliferative and immunomodulatory activities. Infection via a variety of routes has been shown to induce different subtype profiles. IFN-α subtypes bind to the same receptors, activate common signaling pathways and have been expected to have similar immunological functions. All IFN-α subtypes have anti-viral activities, by definition, although their absolute efficacy in this context may vary considerably. In addition, many other biological properties have been described, but with varying potencies, including immunomodulatory and anti-proliferative activities. The pleiotropic effects appear to be due to differential interaction with the receptor chains and signaling through different intracellular pathways to an array of effector molecules.

The Type I IFN receptor consists of two chains, IFNR1 and IFNR2. There is a range of binding affinities for each of the 12 IFN-α subtypes with the different receptor chains. IFNα-14 has one of the highest affinities for both of the two interferon receptors, which is why it is so active compared to the other 11 subtypes. IFNα-6 also has high affinity for the two interferon receptors.

IFN-α may have a key role in the regulation of the Th1 and Th17 responses. It has been shown that IFN-α treatment promotes Th1 cell differentiation indirectly (largely via IFN-γ), but also suppresses Th2 cell development through the suppression of IL4 and IL13 gene expression. IFN-α therefore is able to re-establish a Th1/Th2 population balance in diseases and infections that promote a Th2 cell imbalance. In recent years, it became evident that besides its anti-viral effects, several immunomodulatory functions are exerted by IFN-α. IFN-α can impact on dendritic cell differentiation and controls the expression of various pro-inflammatory cytokines such as IL8 or IL18 and induces several anti-inflammatory mediators such as IL receptor antagonist (IL1Ra), soluble TNF receptor p55, IL10 and IL18 binding protein. However, the mechanisms of actions of IFN-α, and in particular individual IFN-α subtypes, are still only partly understood.

Psoriasis is an autoimmune disease characterised by abnormal patches on the patient's skin. These abnormal patches are typically red, itchy and scaly. The patches normally appear on the elbows, knees, lower back or scalp but can appear anywhere on the body. Psoriasis affects around 125 million people worldwide. Psoriasis is associated with an increased risk of psoriatic arthritis, lymphomas, cardiovascular disease, Crohn's disease and depression. Psoriatic arthritis affects up to 30 percent of individuals with psoriasis.

Psoriasis is characterised by an abnormally excessive and rapid growth of the epidermal layer of the skin. Skin cells are replaced every 3-5 days in psoriasis rather than the usual 28-30 days. These changes are believed to stem from the premature maturation of keratinocytes induced by an inflammatory cascade in the dermis involving dendritic cells, macrophages and T cells. These immune cells (Th17 lymphocytes and macrophages) move from the dermis to the epidermis and secrete inflammatory chemical signals (cytokines), especially tumour necrosis factor-alpha (TNF-α), IL17A, IL17F and IL22. These secreted inflammatory signals stimulate keratinocytes to proliferate and produce chemokines—CXCL1, CXCL5 and CXCL8 (IL8). These cytokines are chemo-attractive to neutrophils, basophils and mast cells and they migrate into the keratinocyte layer. The neutrophils, basophils and mast cells then release the contents of their granules resulting in many of the characteristics of psoriasis.

There is currently no cure for psoriasis; however, various treatments can help control the symptoms. These treatments include corticosteroid creams, vitamin D3 analogue creams, ultraviolet light and immune system suppressing medications, such as methotrexate. About 70-80 percent of psoriasis cases appear in mild-to-moderate form and usually can be managed with creams and/or phototherapy. However, existing topical medications have limited efficacy and are not suitable for long-term use. There have been no new topical medications to treat psoriasis in over twenty-five years. In recent years, research has been focused on systemic agents for the treatment of moderate-to-severe psoriasis e.g. anti-IL17A, TNF-α monoclonal antibody constructs. These new systemic biological drugs are effective but may cause a range of harmful side-effects associated with reduced ability to fight infection. There is a significant unmet need for an effective, safe, topical treatment that can be used to treat the majority of psoriasis patients.

Atopic dermatitis (also known as atopic eczema) is a type of inflammation of the skin that can result in red, swollen and cracked skin. It is a Th2-associated disease and involves increases in IL-3, 4, 5, 13, 17, 22, 31. There are also significant increases in chemokines such as G(M)CSF, CXCL1, 5, and 8 which are involved in granulocyte proliferation and attraction.

### SUMMARY OF THE INVENTION

The present inventor submits that it would be desirable to develop an improved immunotherapeutic approach for the treatment and/or prophylaxis of psoriasis and atopic dermatitis. Since psoriasis results from over-reactivity of Th17 cells and a corresponding overproduction of certain cytokines, a medication that is able to modify and balance a misdirected Th17 response and overproduction of related cytokines would be beneficial in treating psoriasis. Such a medication would further be suitable to treat diseases and conditions where an exaggerated Th17 response plays a role, for example as in atopic dermatitis

The inventor submits that there is a need to provide a topical treatment that can turn off the cytokines/chemokines in the keratinocyte layer that are chemotactic to neutrophils and basophils/mast cells that cause psoriasis in the skin and/or atopic dermatitis.

The present invention relates to compositions and methods for preventing or treating psoriasis, and conditions where an exaggerated Th-17 response plays a detrimental role, such as inflammatory responses and autoimmune. The invention further extends to the use of the compositions of the invention in the treatment and/or prophylaxis of psoriasis.

Following extensive experimentation, the inventor of the present invention has surprisingly discovered that administering IFN-α14, for example SEQ ID NO:1 or a variant or fragment thereof, as described herein results in the suppression or inhibition of various cytokines associated with the immune response in psoriasis or atopic dermatitis. The inventor unexpectedly determined that IFN-α14 can interact directly to turn off the cytokines in the keratinocyte layer that are chemotactic to neutrophils and basophils/mast cells that cause psoriasis in the skin. The inventor demonstrated that IFN-α14 inhibits these chemokines, even under the influence of TNF-α. Surprisingly, this effect is demonstrated when IFN-α14 is administered topically. IFN-α14 is a large molecule of 17,000 Daltons and it was unexpected that this molecule would pass through the skin. What was more surprising was that the inventor unexpectedly found that the effect of IFN-α14 on chemokines in keratinocytes was observed when provided topically. The inventor considers that the topical effects of IFN-α14 are more selective and useful for psoriasis compared to the more indiscriminate effect when IFN-α14 is provided to whole blood—both in the pleiotropic affect and the fact more tissues are brought into contact with the IFN-α14.

The inventor has also established that a recombinant IFN-hybrid molecule known herein as HYBRID 1

CDLPQTHSLGNRRALILLGQMGRISPFSCL
KDRHDFRIPQEEFDGNQFQKAQAISVLHEM
MQQTFNLFSTENSSAAWEQTLLEKFSIELF
QQMNDLEACVIQEVGVEETPLMNEDSILAV
RKYFQRITLYLIERKYSPCAWEVVRAEIMR
SLSFSTNLQKRLRRKD

(SEQ ID NO: 2) also has a high binding affinity to the interferon receptors, and will demonstrate an effect on chemokines involved with psoriasis or atopic dermatitis, in particular to turn off or inhibit chemokines in the keratinocyte layer that are chemotactic to neutrophils and basophils/mast cells that cause psoriasis or Atopic dermatitis in the skin.

Interferon subtypes IFN-α10 and IFN-α14 and hybrids thereof are discussed in PCT Publication Number WO2014/037717 and PCT Publication Number WO2015/136287. In particular IFN-α10-IFN-α14 hybrids are disclosed that contain sequences characteristic of the IFN-α10 and IFN-α14 subtype binding sites based on a consensus backbone sequence of all 12 alpha-interferons. Whilst not wishing to be bound by theory, the inventor believes that proteins comprising the amino acid sequence of IFN-α10 have greater affinity to interferon receptor 2 (IFNR2) and proteins comprising the amino acid sequence of IFN-α14 have greater affinity to interferon receptor 1 (IFNR1). Thus, substitution of a protein comprising an IFN-α10 amino acid sequence with amino acids of IFN-α14 which allow binding to interferon receptor 1 or substitution of a protein comprising an IFN-α14 amino acid sequence with amino acids of IFN-α10 which allow binding to interferon receptor 2 is considered to provide a IFN-α10 IFN-α14 hybrid protein which should have stronger binding affinity to both interferon receptors 1 and 2 than IFN-α10 or IFN-α14 alone. By including the primary interferon receptor binding sites of IFN-α10 and IFN-α14 is meant that the hybrid comprises amino acids selected from IFN-α10 and substituted into an IFN-α14 amino acid sequence to improve the ability of an IFN-α14 subtype to bind to an interferon receptor 2 and/or that the hybrid comprises amino acids selected from IFN-α14 and substituted into an IFN-α10 amino acid sequence to improve the ability of an IFN-α10 subtype to bind to an interferon receptor 1.

Suitably, several amino acid substitutions of protein comprising an IFN-α10 amino acid sequence with amino acids of IFN-α14 determined to be involved in binding to interferon receptor 1 may enhance the binding of the protein to interferon receptor 1. Suitably, an amino acid substitution of protein comprising an IFN-α14 amino acid sequence with amino acids of IFN-α10 determined to be involved in binding to interferon receptor 2 may enhance the binding of the protein to interferon receptor 2.

In embodiments the IFN-α10-IFN-α14 hybrid can substantially have the amino-acid sequence of IFN-α10, but be modified in a region between amino residues 80 to 150, or suitably between amino acid residues 84 to 144, or suitably amino acid residues 92 to 115 or suitably between amino acid residues 90 to 110, (utilizing the numbering of the IFN-α10 sequence) to provide the amino acids provided by the IFN-α14 sequence. It is considered the amino acid residues in these regions or parts of these regions provide for the binding of IFN-α14 to interferon receptor 1. In particular, the hybrid sequence may include at least one, at least two, at least three, at least 4, at least 5, at least 6, at least 7, at least 8, at least 9, at least 10 or at least 11 modifications of the IFN-α10 sequence to provide the corresponding residues of the IFN-α14 sequence or a conserved mutation thereof. In embodiments, eleven modifications are provided as indicated by the amino acids noted in bold

(SEQ ID NO: 3)
CDLPQTHSLGNRRALILLGQMGRISPFSCLKDRHDFRIPQEEFDGNQFQK

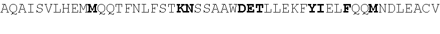

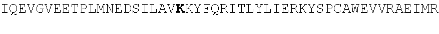

SLSFSTNLQKRLRRKD

In embodiments, the IFN-α10-IFN-α14 hybrid sequence may include at least one mutation selected from amino acids at positions 94, 101, 102, 109 or 144, preferably at least two mutations selected from amino acids at positions 94, 101, 102, 109 or 144, more preferably at least three mutations selected from amino acids at positions 94, 101, 102, 109 or 144, more preferably at least four mutations selected from amino acids at positions 94, 101, 102, 109 or 144 or more preferably at least five mutations selected from amino acids at positions 94, 101, 102, 109 or 144. In alternative embodiments, IFN-α14 can be utilised as a backbone structure of the hybrid and the residues which differ between the IFN-α10 and IFN-α14 sequences at the N and C terminal regions of the sequences can be provided in the hybrid sequence as those present in the IFN-α10 sequence. Suitably at least 1, at least 2, at least 3, at least 4, at least 5, at least 6, at least 7, at least 8, at least 9, at least 10 or at least 11 substitutions of the IFN-α14 N-terminal sequence may be made to provide the hybrid sequence to provide residues from IFN-α10 at those amino acid positions wherein the amino acids are not shared/common between IFN-α10 and IFN-α14. Suitably, at least 1, at least 2, or 3 substitutions are provided at the IFN-α14 C terminal sequence to provide residues from IFN-α10 to the hybrid sequence at those amino acid positions which are not shared/common between IFN-α10 and IFN-α14. In embodiments at least 1, at least 2, at least 3, at least 4, at least 5, at least 6, at least 7, at least 8, at least 9, at least 10 or at least 11 substitutions from the N-terminal sequence and at least 1, at least 2, or 3 substitutions from the C-terminal sequence of the IFN-α14 are made to provide residues from IFN-α10 to the hybrid at those amino acid positions which have amino acids that are not shared/common between IFN-α10 and IFN-α14.

In embodiments, the hybrid comprises or consists of an amino acid sequence SEQ ID NO: 2 or a functionally active fragment or variant thereof.

By functionally active is meant an IL-α10 IL-α14 hybrid polypeptide comprising the primary interferon binding sites of IFN-α10 and IFN-α14 wherein the administration of peptide to a subject or expression of peptide in a subject promotes enhancement of Th1 mediated immune response and suppression of a Th2/Th17 mediated immune response. Further, functional activity may be indicated by the ability of a hybrid peptide to enhance a Th1 mediated immune response and to suppress a Th2/Th17 mediated response.

A fragment can comprise at least 50, preferably 100 and more preferably 150 or greater contiguous amino acids from SEQ ID NO: 1 or 2 and which is functionally active. Suitably, a fragment may be determined using, for example, C-terminal serial deletion of cDNA. Said deletion constructs may then be cloned into suitable plasmids. The activity of these deletion mutants may then be tested for biological activity as described herein.

By variant is meant an amino acid sequence which is at least 70% homologous to SEQ ID NO: 1 or 2, more preferably at least 80% homologous to SEQ ID NO: 1 or 2, more preferably at least 90% homologous to SEQ ID NO: 1 or 2, even more preferably at least 95% homologous to SEQ ID NO: 1 or 2, even more preferably at least 96% homologous to SEQ ID NO: 1 or 2, even more preferably at least 97% homologous to SEQ ID NO: 1 or 2 and most preferably at least 98% homology with SEQ ID NO: 1 or 2. A variant encompasses a polypeptide sequence of SEQ ID NO: 1 or 2 which includes substitution of amino acids, especially a substitution(s) which is/are known for having a high probability of not leading to any significant modification of the biological activity or configuration, or folding, of the protein. These substitutions, typically known as conserved substitutions, are known in the art. For example the group of arginine, lysine and histidine are known interchangeable basic amino acids. Suitably, in embodiments amino acids of the same charge, size or hydrophobicity may be substituted with each other. Suitably, any substitution may be selected based on analysis of amino acid sequence alignments of interferon alpha subtypes to provide amino acid substitutions to amino acids which are present in other alpha subtypes at similar or identical positions when the sequences are aligned. Hybrids, and variants and fragments thereof may be generated using suitable molecular biology methods as known in the art.

The inventor also considers that there is some relevance for the use of IFN-α14 or HYBRID 1 (SEQ ID NO:2) as a topical treatment in relation to the sub-epidermis layers of the skin. Without wishing to be bound by theory it is considered a portion of the IFN-α14 or HYBRID 1 can pass through the skin to the lower dermis layer, where there are many leucocytes, especially Th17 that make IL17A, IL17F and IL22. IL17A and IL17F stimulate macrophages to make TNF-α, which is the major mediator that causes the keratinocytes to release granulocyte attracting chemokines such as CXCL1, CXCL5 and CXCL8.

The inventor has surprisingly discovered that administration of IFN-α14 or HYBRID 1, in particular SEQ ID NO:1 or 2 or a variant or fragment thereof, as a topical treatment results in a greater reduction or inhibition of CXCL1, CXCL8 (IL8), CXCL-5 and CCL20 in keratinocytes compared to previous topical medications. In addition, the inventors have determined that very low doses of IFN-α14 or HYBRID 1, for example up to 5×10³ IU/ml or 5×10⁴IU/ml topical cream can be used.

The inventor also indicates that as IFN-α14, in particular SEQ ID NO:1 or a variant or fragment thereof, diffuses down into the lower dermis layer it can also turn off or inhibit TNF-α which also results in inhibition of chemokines such as IL17, e.g. IL17A, IL17B, IL17F and/or IL22.

This has led to the identification by the inventor of improved therapeutic compositions which have utility in the treatment and/or prophylaxis of psoriasis and diseases and conditions where an exaggerated Th17, 22 response plays a role such as atopic dermatitis.

Accordingly a first aspect of the present invention provides a method for the treatment and/or prophylaxis of psoriasis or atopic dermatitis, said method comprising the step of:
(i) administering to a subject in need thereof a therapeutically effective amount of an interferon alpha subtype, wherein the interferon alpha subtype is IFN-α14, HYBRID 1 or a combination of IFN-α14 and HYBRID 1.

In embodiments, the interferon alpha subtype IFN-α14 comprises or consists of an amino acid sequence SEQ ID NO:1 or a functionally active fragment or variant thereof.

In embodiments, the interferon alpha subtype HYBRID 1 comprises or consists of an amino acid sequence SEQ ID NO:2 or a functionally active fragment or variant thereof.

In embodiments, the method of administration is topical administration. In embodiments the method of administration is sub-lingual. Without wishing to be bound by theory in both methods of administration it is considered that a concentration of IFN-α14 and HYBRID 1 would be provided such that systemic effects of interferon are not induced. Thus, the chemokine and interleukin effects can be achieved without causing (or only minimally causing) antivral or anti-proliferative effects.

It would be considered this administration is distinguished from systemic delivery of interferons in the art which have provided pharmacological doses. Such pharmacological doses would activate anti-viral/bacterial properties of such interferons (for example as would have been observed following administration of IFNalpha2c in the art)—causing side effects and abrogating the low concentration-associated immune regulation effects observed by the inventors following topical administration. Typically, topical doses may be 100-1000×less than systemic doses and allow control of immune response in the skin compartment only.

In embodiments, the therapeutically effective amount of the interferon alpha subtype is alow dose (up to 5×10⁴IU units or 5×10³IU units/ml). In embodiments, the therapeutically effective amount of the interferon alpha subtype is lower than current systemic treatments for psoriasis or other conditions.

In embodiments, the interferon alpha subtype is administered in a dose of 5 IU/ml, 10 IU/ml, 50 IU/ml, 1×10²IU/ml, 1×10³IU/ml, 1×10⁴IU/ml, 1×10⁵IU/ml or 1×10⁶IU/ml.

The inventors have elucidated that interferon alpha subtypes cause a varied response both from each other, and dependent on dose (high dose leading to systemic—anti-viral and anti-proliferative effects) and low dose-chemokine and interleukin effects at a non-systemic level and that response can vary dependent of tissue.

In embodiments, the interferon alpha subtype is administered in a dose of 0.1 mg to 1 mg, 1 mg to 3 mg, 3 mg to 5 mg or 5 mg to 10 mg. For example, in human topical applications 5×10⁴ IU/ml cream or less may be used. In animals, e.g. dog, sublingual use may be 104 IU/Kg, for example in 1 ml PBS.

In embodiments, the interferon alpha subtype is topically administered once a day, twice a day, three times a day or four times a day. Typically for sublingual administration, the dose would be provided once a day.

In embodiments, the interferon alpha subtype IFN-α14 and HYBRID 1 interacts directly to turn off or inhibit the cytokines/chemokines in the keratinocyte layer. In embodiments, the interferon alpha subtype IFN-α14 and HYBRID 1 interacts directly to turn off the cytokines in the keratinocyte layer that are chemotactic to neutrophils and basophils/mast cells that cause psoriasis in the skin. In embodiments, the interferon alpha subtype passes through the skin to the lower dermis layer where it effects chemokine production.

In embodiments atopic dermatitis can be localised to particular locations on the body, for example the bends of the arms, legs, face, neck, eyelids, wrist, finger, knuckles, ankles, feet and/or hands. In embodiments atopic dermatitis can affect the whole or substantially the whole body. This may be observed particularly for animals, for example in dogs where atopic dermatitis may cause them to scratch all over their body.

In certain embodiments, the psoriasis can be mild, mild-to-moderate, moderate, moderate-to-severe or severe psoriasis.

Typically, the subject is a mammal, in particular a human. In embodiments the subject can be an animal, for example, but not limited to a companion animal such as a dog.

In certain embodiments, the subject can be suffering from a condition where suppression of a Th17-mediated immune response is desired. In certain embodiments, the subject can be suffering from psoriasis. In embodiments, the subject can be suffering from Atopic Dermatitis.

According to a second aspect of the present invention, there is provided an interferon alpha subtype, wherein the interferon alpha subtype is IFN-α14, or HYBRID 1 or a combination of IFN-α14 and HYBRID 1 for use in the treatment and/or prophylaxis of psoriasis or atopic dermatitis or a condition where suppression of a Th17-mediated immune response is desired.

In embodiments, the interferon alpha subtype IFN-α14 comprises or consists of an amino acid sequence SEQ ID NO:1 or a functionally active fragment or variant thereof.

In embodiments, the interferon alpha subtype HYBRID 1 comprises or consists of an amino acid sequence SEQ ID NO:2 or a functionally active fragment or variant thereof.

In certain embodiments, the interferon alpha subtype will be administered topically.

In certain embodiments the interferon alpha subtype may be administered sublingually. This may be particularly advantageous for veterinary treatments.

In embodiments, the interferon alpha subtype is administered at a low dose as discussed herein. In embodiments, the interferon alpha subtype is administered at a very low dose. In embodiments, the therapeutically effective amount of the interferon alpha subtype is lower than current systemic treatments for psoriasis.

In embodiments, the the interferon alpha subtype can be administered in a dose of 5 IU/ml, 10 IU/ml, 50 IU/ml, 1×10²IU/ml, 1×10³IU/ml, 1×10⁴IU/ml, 1×10⁵IU/ml or 1×10⁶IU/ml.

In embodiments, the the interferon alpha subtype can be administered in a dose of 0.1 mg to 1 mg, 1 mg to 3 mg, 3 mg to 5 mg or 5 mg to 10 mg.

In embodiments, the the interferon alpha subtype can be administered once a day, twice a day, three times a day or four times a day. Suitably, in sublingual administration, a single does may be provided each day.

In certain embodiments, the psoriasis can be mild, mild-to-moderate, moderate, moderate-to-severe or severe psoriasis. The severity of psoriasis may be assessed by PASI scores. This gives a figure to the coverage of the lesions on the patient. A 75% reduction in the Psoriasis Area and Severity Index (PASI) score (PASI 75) is the current benchmark of primary endpoints for most clinical trials of psoriasis.

According to a third aspect of the present invention, there is provided use of an interferon alpha subtype, wherein the interferon-alpha subtype is IFN-α14, HYBRID 1, or a combination of IFN-α14 and HYBRID 1, in the preparation of a medicament for the treatment and/or prophylaxis of psoriasis or a condition where suppression of a Th17-mediated immune response is desired. Suitably a condition may be atopic dermatitis.

According to a further aspect of the present invention, there is provided a composition comprising an interferon alpha subtype, wherein the interferon alpha subtype is IFN-α14, HYBRID 1 or a combination of IFN-α14 and HYBRID 1, for use in the treatment and/or prophylaxis of psoriasis or a condition where suppression of a Th17-mediated immune response is desired.

According to a further aspect of the present invention, there is provided a pharmaceutical composition comprising an interferon alpha subtype, wherein the interferon alpha subtype is IFN-α14, HYBRID 1 or a combination of IFN-α14 and HYBRID 1, for use in the treatment and/or prophylaxis of psoriasis or a condition where suppression of a Th17-mediated immune response is desired.

According to a further aspect of the present invention, there is provided an interferon alpha subtype, wherein the interferon subtype is IFN-α14, HYBRID 1 or a combination of IFN-α14 and HYBRID 1, for use in modulating an immune response.

In embodiments of the aspects of the invention outlined above, the interferon alpha subtype IFN-α14 comprises or consists of an amino acid sequence SEQ ID NO:1 or a functionally active fragment or variant thereof.

In embodiments of the aspects of the invention outlined above, the interferon alpha subtype HYBRID 1 comprises or consists of an amino acid sequence SEQ ID NO:2 or a functionally active fragment or variant thereof.

In embodiments of the aspects of the invention outlined above, the composition or pharmaceutical composition is administered topically.

In embodiments of the aspects of the invention outlined above, the interferon alpha subtype is administered at a low dose. In embodiments, the interferon alpha subtype is administered at a very low dose. In embodiments, the therapeutically effective amount of the interferon alpha subtype is lower than current systemic treatments for psoriasis.

In embodiments of the aspects of the invention outlined above, the the interferon alpha subtype is administered in a dose of IU/ml, 10 IU/ml, 50 IU/ml, 1×10²IU/ml, 1×10³IU/ml, 1×10⁴IU/ml, 1×10⁵IU/ml or 1×10⁶IU/ml.

In embodiments of the aspects of the invention outlined above, the the interferon alpha subtype is administered in a dose of 0.1 mg to 1 mg, 1 mg to 3 mg, 3 mg to 5 mg or 5 mg to 10 mg.

In embodiments of the aspects of the invention outlined above, the the interferon alpha subtype is administered once a day, twice a day, three times a day or four times a day.

In certain embodiments of the aspects of the invention outlined above, the psoriasis can be mild, mild-to-moderate, moderate, moderate-to-severe or severe psoriasis.

In certain embodiments of the aspects of the invention outlined above, the IFN-α subtype comprises, consists of or is IFN-α14 such as a fusion protein, or recombinant protein or the like and in particular which comprises or consists of the amino acid sequence SEQ ID NO:1 or a variant or fragment thereof. In embodiments the IFN-α14 can be glycosylated.

In certain embodiments of the aspects of the invention outlined above, the IFN-α subtype comprises, consists of or is HYBRID 1 such as a fusion protein, or recombinant protein or the like and in particular which comprises or consists of the amino acid sequence SEQ ID NO:2 or a variant or fragment thereof.

In a further aspect of the invention there is provided a recombinant polypeptide comprising or consisting of SEQ ID NO:1 or a fragment or variant thereof. The invention extends to nucleic acid sequences derived from the amino acid sequence SEQ ID NO:1.

In a further aspect of the invention there is provided a recombinant polypeptide comprising or consisting of SEQ ID NO:2 or a fragment or variant thereof. The invention extends to nucleic acid sequences derived from the amino acid sequence SEQ ID NO:2.

### DETAILED DESCRIPTION OF THE INVENTION

The inventor of the present invention has surprisingly discovered that administering IFN-α14, for example SEQ ID NO:1 or a variant or fragment thereof, as described herein results in the suppression or inhibition of various cytokines associated with the immune response in psoriasis. Surprisingly, this effect is enhanced when the IFN-α14 is administered topically.

SEQ ID NO:1 is IFNα-14 and can be defined as follows:

CNLSQTHSLNNRRTLMLMA QMRRISPFSCLKDRHDFEFP
QEEFDGNQFQKAQAISVLHE MMQQTFNLFSTKNSSAAWDE
TLLEKFYIELFQQMNDLEAC VIQEVGVEETPLMNEDSILA
VKKYFQRITLYLMEKKYSPC AWEVVRAEIMRSLSFSTNLQ
KRLRRKD

SEQ ID NO:2 is HYBRID-1 and can be defined as follows:

CDLPQTHSLGNRRALILLGQMGRISPFSCL
KDRHDFRIPQEEFDGNQFQKAQAISVLHEM
MQQTFNLFSTENSSAAWEQTLLEKFSIELF
QQMNDLEACVIQEVGVEETPLMNEDSILAV
RKYFQRITLYLIERKYSPCAWEVVRAEIMR
SLSFSTNLQKRLRRKD

In particular, the inventor has discovered that IFN-α14, in particular SEQ ID NO:1 or HYBRID 1 (SEQ ID NO:2) or a variant or fragment thereof, targets specific cytokines in the keratinocyte layer associated with psoriasis (e.g. CXCL-1,5,8, but not CCL-1, 5, IL-6. The immune response in psoriasis involves a IL23/Th17/IL-17A axis. IL23 is produced from dendritic cells or monocytes. Th17 lymphocyte is activated to release IL17A, IL17B and IL17F. IL17 further stimulates macrophages to release large amounts of TNF-α. This causes the release of CXCL8 (IL8), CXCL5, CCL-20 and CXCL1 from keratinocytes. This attracts neutrophils and basophils/mast cells that then release agents that cause psoriatic plaques. In addition, TNF-α is a major contributor to plaque development. IL22 is an activator of inflammation and inhibits keratinocyte terminal differentiation. In recent years, new systemic drugs have been developed to target IL23, IL17A or TNF-α individually. The inventors have demonstrated that the natural molecule IFNα-14, in particular SEQ ID NO:1 or SEQ ID NO:2 or a variant or fragment thereof, eliminates or turns off CXCL1, CXCL8 (IL8), CXCL5 and CCL20 in keratinocytes at very low doses. HYBRID 1 provides the same functional effects as IFN-α14 at the same does on the IL-17 pathway and associated chemokines. Moreover, whilst the inventors have determined that unlike IFN-α14, it is a poor activator of NK-cells (see FIG. 25). This can be advantageous as it means HYBRID 1 will have a “better side effects” profile. The present inventor has also determined that when IFNα-14, in particular SEQ ID NO:1 or a variant or fragment thereof, moves down into the dermis layer, it targets IL23, IL17A, IL17F and TNF-α IL17F simultaneously. These findings can be applied to provide an improved method and improved composition for treating and/or preventing psoriasis.

The inventor considers IFN-αl4 and HYBRID 1 act on:
(i) CXCL1: induces inflammation, attracts neutrophils and causes the release of their destructive enzymes;
(ii) CXCL8 (IL8): a chemokine from keratinocytes that is an attractant for neutrophils, basophils and mast cells causing release of many tissue damaging substances;
(iii) CXCL5: well known to have chemotactic and activating functions on neutrophils, mainly during acute inflammatory responses. It also maintains neutrophil homeostasis;
(iv) IL6: a growth factor from keratinocytes commonly associated with stress and fever. IL6 is an acute phase reactant and can be both pro- and anti-inflammatory. IL6 can act before IL17 production by inhibiting Th17 cell generation and after by suppressing the production of IL6 which increases keratinocyte proliferation;
(v) TNF-α: a major contributor to plaque development and activates unwanted chemokine production from keratinocytes;
(vi) IL17 and IL23: clinically validated as having the central role in the pathogenesis of psoriasis; and
(vii) IL-22: inflammation and inhibits keratinocyte terminal differentiation and replicates many of the chemokine stimulating activities of IL-17.

CXCL8 (IL8) is the primary cytokine involved in the recruitment of neutrophils to the site of damage or infection; a process called chemotaxis. A number of variables are essential for the successful chemotaxis of neutrophils, including the increased expression of high affinity adhesion molecules to secure the neutrophil to the endothelium near the affected site (and is, therefore, not washed away into the circulatory system), and that the neutrophil can digest its way through the basement membrane and the extracellular matrix (ECM) to reach affected site. CXCL8 plays a key role in inducing the cell signalling necessary to bring about these changes. Firstly, at the site of infection histamine release causes vasodilation of the capillaries near the injured area which slows down the blood flow in the region and encourages leukocytes, such as neutrophils, to come closer to the endothelium, and away from the centre of the lumen where the rate of blood flow is highest. Once this occurs weak interactions are made between the selectins expressed on the neutrophil and endothelial cells (expression of which is also increased through the action of CXCL8 and other cytokines).

The inventor has discovered that administration of IFN-α14, in particular SEQ ID NO:1 or a variant or fragment thereof, results in a 10%, preferably a 20%, preferably a 30%, preferably a 40%, preferably a 50%, preferably a 60%, preferably a 70%, preferably a 80% and more preferably a 87% greater reduction of IL-17 (IL-17A, IL-17B or IL-17F) compared to previous topical medications. It is considered HYBRID 1 will have the same effects.

The inventor has surprisingly discovered that administration of IFN-α14, in particular SEQ ID NO:1 or a variant or fragment thereof, enables IL-22 to inhibit α/β and γ/δ T lymphocyte synthesis by 50%, preferably 60%, preferably 70%, preferably 76%, preferably greater than 76%, preferably 80%, preferably 90%, and more preferably 95%. Again, HYBRID 1 is considered to provide the same effects.

The inventor has surprisingly discovered that administration of IFN-α14, in particular SEQ ID NO:1 or a variant or fragment thereof, results in the suppression of CXCL1, CXCL8 (IL-8), CXCL-5 or CCL-20 in keratinocytes by 50%, preferably by 60%, preferably by 70%, preferably by 80%, preferably by 90%, preferably by 91%, preferably by 92%, preferably by 93%, preferably by 94%, preferably by 95%, preferably by 96%, preferably by 97%, and more preferably by 98%.

The inventor has surprisingly discovered that administration of IFN-α14, in particular SEQ ID NO:1 or a variant or fragment thereof, results in the suppression of CXCL1, CXCL8 (IL8), CXCL5 or CCL20 in keratinocytes at low doses as discussed herein. Again, HYBRID 1 shows similar functional effects.

Treatment of the present invention can be administered in a dose of 5 IU/ml, 10 IU/ml, 50 IU/ml, 1×10²IU/ml, 1×10³IU/ml, 1×10⁴IU/ml, 1×10⁵IU/ml or 1×10⁶IU/ml.

The treatment of the present invention can be administered in a dose of 0.1 mg to 1 mg, 1 mg to 3 mg, 3 mg to 5 mg or 5 mg to 10 mg.

The treatment of the present invention can be topically administered once a day, twice a day, three times a day or four times a day.

In addition, the inventor has discovered that the administration or use of IFN-α14, in particular SEQ ID NO:1 or HYBRID 1 (SEQ ID NO:2) or a variant or fragment thereof, results in the full or partial inhibition of CXCL1 and/or the full or partial inhibition of CXCL8 (IL8) and/or the full or partial inhibition of CXCL5 and/or the full or partial inhibition of CCL20 in keratinocytes

In addition, the inventor has discovered that the administration or use of IFN-α14, in particular SEQ ID NO:1 or HYBRID 1 (SEQ ID NO:2) or a variant or fragment thereof, results in the full or partial inhibition of IL17 and/or the full or partial inhibition of IL22 and/or the full or partial inhibition of IL23 and/or and/or the full or partial inhibition of IL6 and/or the full or partial inhibition of TNF-α in dermal layers.

Moreover, the inventor has surprisingly discovered that the topical administration of IFN-α14 or HYBRID 1, in particular SEQ ID NO:1 or SEQ ID NO:2 or a fragment or variant thereof, can result in the targeting of the relevant cytokines in the keratinocyte layer as discussed herein. The present invention provides a superior topical treatment that is safe and effective in mild, moderate and severe psoriasis. This treatment demonstrates a low side-effect profile. Low doses of the medication are required and the natural product, IFN-α14, or HYBRID 1 displays no cytotoxicity in-vitro at even the highest concentration (1×10⁸ IU/ml).

The inventor has demonstrated on keratinocytes from normal human skin cultures that were activated with TNF-α to induce chemokine (Il8) secretion, that IFNα-14, in particular SEQ ID NO:1 or a variant or fragment thereof, suppressed IL8 secretion directly by >80%. In addition, the inventor demonstrated that when tested on biopsies from normal human skin induced into a psoriatic state, the addition of IFNα-14, in particular SEQ ID NO:1 or a variant or fragment thereof, resulted in strong inhibition of the secretion of IL17A, IL17F and IL22. These results are a clear indication of the potential superiority of IFNα-14, in particular SEQ ID NO:1 or a variant or fragment thereof, over existing systemic biologics.

The inventor, whilst not wishing to be bound by theory, has identified that the topical administration of IFN-α14, in particular SEQ ID NO:1 or a variant or fragment thereof, can be used to treat psoriasis and atopic dermatitis. The inventors have demonstrated that in spite of its relatively high molecular weight, IFNα-14 and HYBRID 1, in particular SEQ ID NO:1 or SEQ ID NO:2 or a variant or fragment thereof, displays good permeation potential through the skin and, therefore, the development of clinically viable formulations that enable delivery of therapeutics doses of this peptide across the skin provides an unexpected approach to treat or prevent psoriasis.

### Definitions

Fragment

A fragment can comprise at least 50, preferably 100 and more preferably 150 or greater contiguous amino acids from SEQ ID NO: 1 or SEQ ID NO:2 and which is functionally active. Suitably, a fragment may be determined using, for example, C-terminal serial deletion of cDNA. Said deletion constructs may then be cloned into suitable plasmids. The activity of these deletion mutants may then be tested for biological activity as described herein. Fragments may be generated using suitable molecular biology methods as known in the art.

Variant

By variant is meant an amino acid sequence which is at least 70% homologous to SEQ ID NO: 1 or SEQ ID NO:2, more preferably at least 80% homologous to SEQ ID NO: 1 or SEQ ID NO:2, more preferably at least 90% homologous to SEQ ID NO: 1 or SEQ ID NO:2, even more preferably at least 95% homologous to SEQ ID NO: 1 or SEQ ID NO:2, even more preferably at least 96% homologous to SEQ ID NO: 1 or SEQ ID NO:2, even more preferably at least 97% homologous to SEQ ID NO: 1 or SEQ ID NO:2, and most preferably at least 98% homology with SEQ ID NO: 1 or SEQ ID NO:2. A variant encompasses a polypeptide sequence of SEQ ID NO: 1 or SEQ ID NO:2 which includes substitution of amino acids, especially a substitution(s) which is/are known for having a high probability of not leading to any significant modification of the biological activity or configuration, or folding, of the protein. These substitutions, typically known as conserved substitutions, are known in the art. For example the group of arginine, lysine and histidine are known interchangeable basic amino acids. Suitably, in embodiments amino acids of the same charge, size or hydrophobicity may be substituted with each other. Suitably, any substitution may be selected based on analysis of amino acid sequence alignments of interferon alpha subtypes to provide amino acid substitutions to amino acids which are present in other alpha subtypes at similar or identical positions when the sequences are aligned. Variants may be generated using suitable molecular biology methods as known in the art.

Subject

As herein defined, a “subject” includes and encompasses mammals such as humans, primates and livestock animals (e.g. sheep, pigs, cattle, horses, donkeys); laboratory test animals such as mice, rabbits, rats and guinea pigs; and companion animals such as dogs and cats.

Treatment/Therapy

The term “treatment” is used herein to refer to any regimen that can benefit a human or non-human animal. The treatment may be in respect of psoriasis and the treatment may be prophylactic (preventative treatment). Treatment may include curative or alleviative effects. Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and/or prophylactic treatment includes amelioration of the symptoms of a particular allergic condition or preventing or otherwise reducing the risk of developing a particular allergic condition. The term “prophylactic” may be considered as reducing the severity or the onset of a particular condition. “Therapeutic” may also reduce the severity of an existing condition.

Administration

The active ingredients used in the present invention in particular the interferon subtype IFN-α14, for example SEQ ID NO: 1 or HYBRID 1 (SEQ ID NO:2), as described herein can be administered separately to the same subject, optionally sequentially, or can be co-administered simultaneously as a pharmaceutical or immunogenic composition. The pharmaceutical composition will generally comprise a suitable pharmaceutical excipient, diluent or carrier selected depending on the intended route of administration.

The active ingredients can be administered to a patient in need of treatment via any suitable route. The precise dose will depend upon a number of factors, as is discussed below in more detail.

One suitable route of administration is topically, e.g. applied directly to the skin.

Pharmaceutical Compositions

As described above, the present invention extends to a pharmaceutical composition for the treatment psoriasis or atopic dermatitis.

Pharmaceutical compositions according to the present invention, and for use in accordance with the present invention, may comprise, in addition to an active ingredient, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example, oral, intravenous, intranasal or via oral or nasal inhalation. The formulation may be a liquid, for example, a physiologic salt solution containing non-phosphate buffer at pH 6.8-7.6, or a lyophilised or freeze-dried powder.

Dose

The composition is preferably administered to an individual in a “therapeutically effective amount” or a “desired amount”, this being sufficient to show benefit to the individual. As defined herein, the term an “effective amount” means an amount necessary to at least partly obtain the desired response, or to delay the onset or inhibit progression or halt altogether the onset or progression of a particular condition being treated. The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials. Prescription of treatment, e.g. decisions on dosage etc., is ultimately within the responsibility and at the discretion of general practitioners, physicians or other medical doctors, and typically takes account of the disorder to be treated, the condition of the individual patient, the site of delivery, the method of administration and other factors known to practitioners. The optimal dose can be determined by physicians based on a number of parameters including, for example, age, sex, weight, severity of the condition being treated, the active ingredient being administered and the route of administration. A broad range of doses may be applicable. Considering oral administration to a human patient, for example, from about 10 g to about 1000 g of agent may be administered per human dose, optionally for 3 to 4 doses. Dosage regimes may be adjusted to provide the optimum therapeutic response and reduce side effects. For example, several divided doses may be administered daily, weekly, monthly or other suitable time intervals or the dose may be proportionally reduced as indicated by the exigencies of the situation.

Unless otherwise defined, all technical and scientific terms used herein have the meaning commonly understood by a person who is skilled in the art in the field of the present invention.

Autoimmune Disease

The term “autoimmune disease” as used herein is understood to mean any disease or condition which is caused by a body's tissues being attacked by its own immune system.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

The present invention will now be exemplified with reference to the following non-limiting figures and examples which are provided for the purpose of illustration and are not intended to be construed as being limiting on the present invention. Other embodiments of this invention will be apparent to those of ordinary skill in the art in view of this description.

### BRIEF DESCRIPTION OF THE FIGURES

FIG. 1 shows a graph demonstrating the effect of IFNα-14 on CXCL8 (IL8) production from human keratinocytes with and without induction with TNF-α.

FIG. 2 shows a graph demonstrating the effect of IFNα-14 on CXCL1 production from human keratinocytes with and without induction with TNF-α.

FIG. 3 shows a graph demonstrating the effect of IFNα-14 on CXCL5 production from human keratinocytes with and without induction with TNF-α.

FIG. 4 shows a graph demonstrating the effect of IFNα-14 on IL6 production from human keratinocytes without induction with TNF-α.

FIG. 5 shows a graph demonstrating the effect of IFNα-14 on CCL2 production from human keratinocytes without induction with TNF-α.

FIG. 6 shows a graph demonstrating the effect of IFNα-14 on CCL5 production from human keratinocytes without induction with TNF-α.

FIG. 7 shows a graph demonstrating the effect of IFNα-14 on CCL20 production from human keratinocytes without induction with TNF-α.

FIG. 8 shows a graph demonstrating the effect of IFNα-14 on IL17A production in normal skin biopsies stimulated into a psoriatic state.

FIG. 9 shows a graph demonstrating the effect of IFNα14 on IL17A production in whole human blood assays.

FIG. 10 shows a graph demonstrating the effect of IFNα-14 on IL17F production in whole human blood assays.

FIG. 11 shows a graph demonstrating the effect of IFNα-14 on IL22 production in whole human blood assays.

FIG. 12 shows a graph demonstrating the effect of IFNα-14 on TNF-α production in whole human blood assays.

FIG. 13 shows a graph demonstrating the effect of IFNα-14 on IL6 production in whole human blood assays.

FIG. 14 shows a graph demonstrating the effect of IFNα-14 on CXCL8 (IL8) production in whole human blood assays.

FIG. 15 shows a graph demonstrating the effect of IFNα-14 on CXCL1 production in whole human blood assays.

FIG. 16 shows the IFN-α14 amino acid sequence.

FIG. 17 shows the HYBRID 1 amino acid sequence.

FIG. 18 shows changes in concentrations of interleukins, chemokines and CD markers following treatment of Human mononuclear cells with human IFN alpha 10/14.

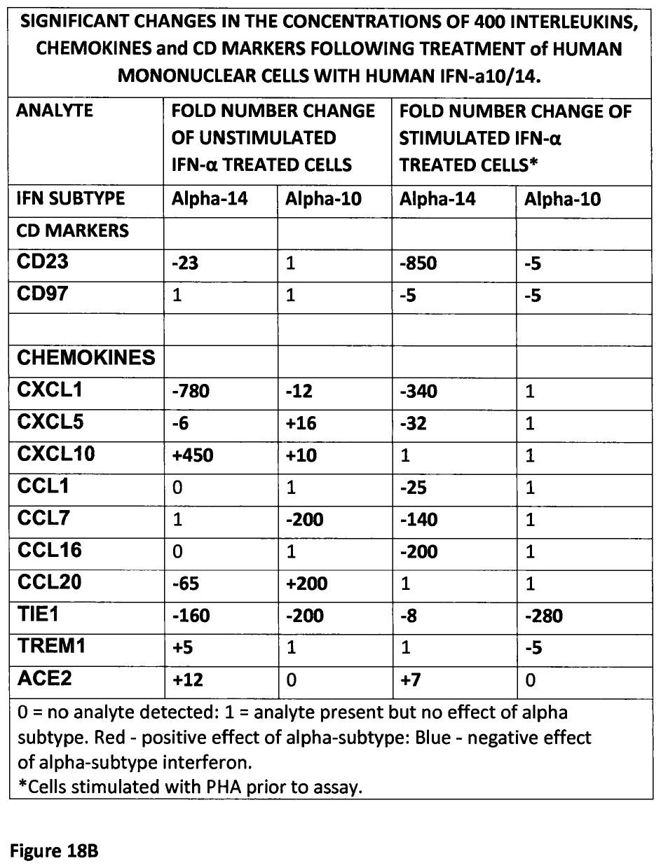

FIG. 19 shows inhibition of canine IL-17A secretion from canine leukocytes with human IFN-α14.

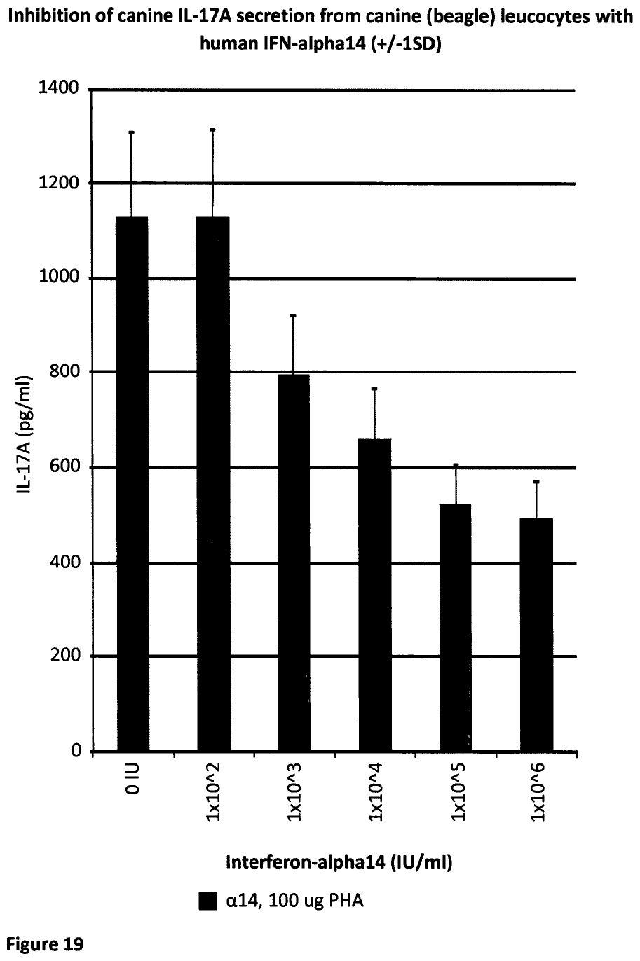

FIG. 20 shows a comparison of effects of the HYBRID 1 and IFN-α14 on production of IL-17A.

FIG. 21 shows a comparison of effects of the HYBRID 1 and IFN-α14 on production of IL-8.

FIG. 22 shows a comparison of effects of the HYBRID 1 and IFN-α14 on production of CXCL-1.

FIG. 23 shows a comparison of effects of the HYBRID 1 and IFN-α14 on production of Interferon gamma.

FIG. 24 shows a comparison of effects of the HYBRID 1 and IFN-α14 on production of Tumour Necrosis Factor alpha.

FIG. 25 shows a comparison of the effects of IFN-α14 with HYBRID 1 on secretion of CXCL-10.

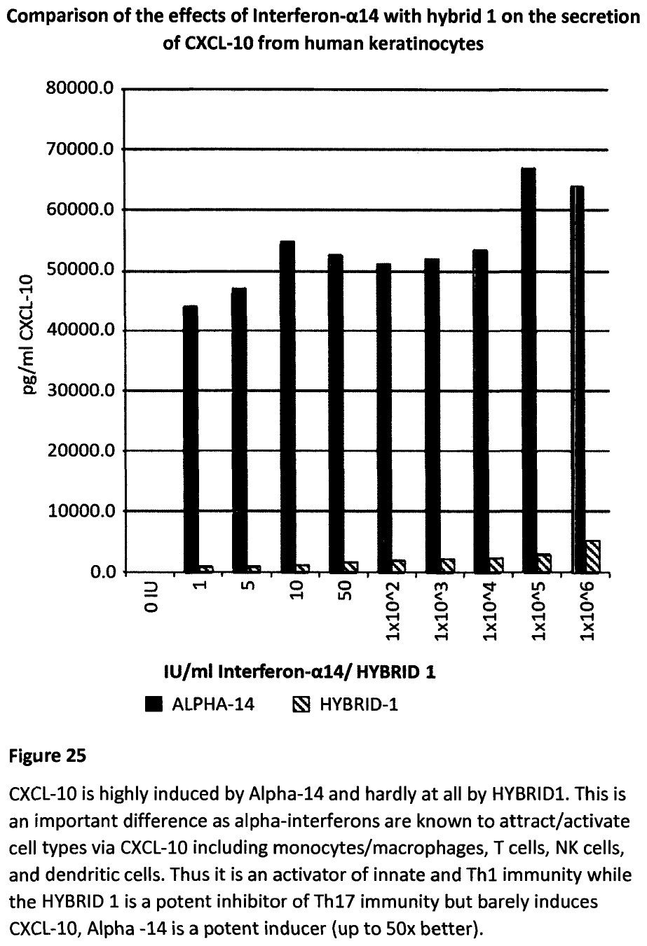

### EXPERIMENTAL DATA

### Experiment 1: The Effect of IFNα-14 on IL-6. CXCL8 (IL8), CXCL1 and CCL2 Production in Ketatinocytes from Normal Human Skin

The inventor tested the effect of IFNα-14 on keratinocytes from normal human skin that were activated with TNF-α to induce chemokine secretion.

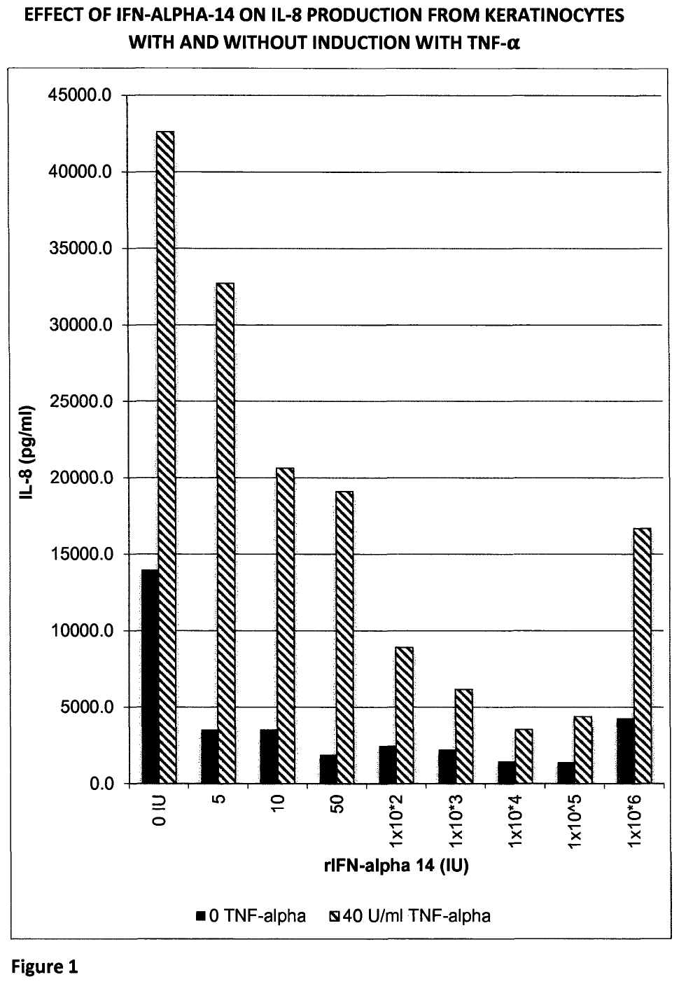

FIG. 1 demonstrates that IFNα-14 suppresses CXC18 (IL8) secretion directly by >80%. CXCL8 (IL-8) is the major chemokine involved in psoriasis and FIG. 1 indicates strong inhibition of CXCL8 (IL8) in the presence of IFNα-14.

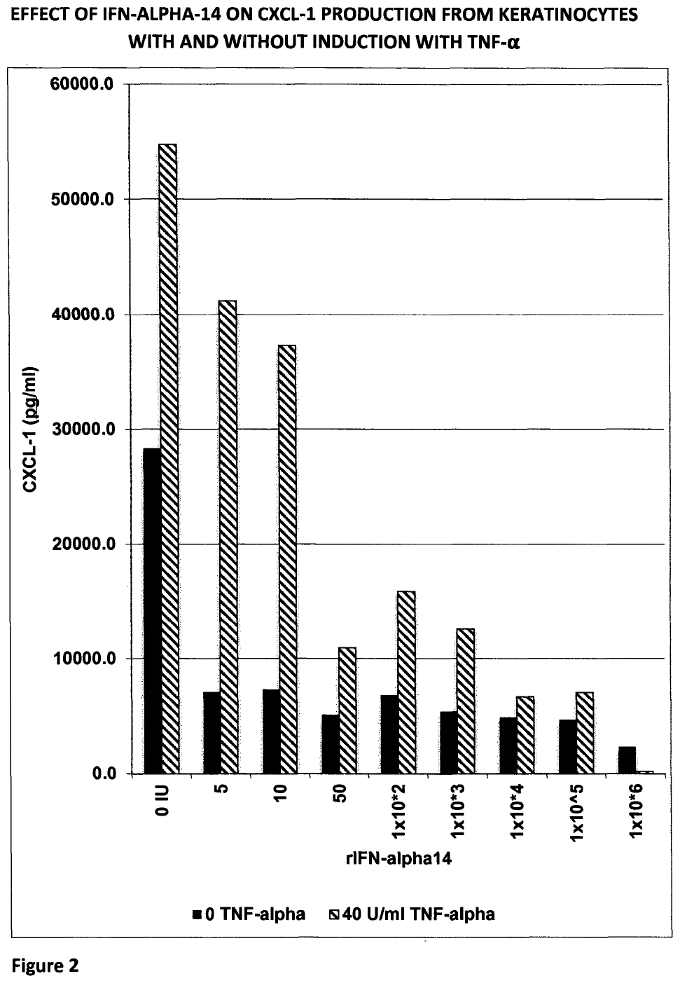

FIG. 2 demonstrates strong inhibition of CXCL1 production. CXCL1 is a member of CXC family, which plays an integral role in recruitment and activation of neutrophils in response to tissue injury and microbial infection.

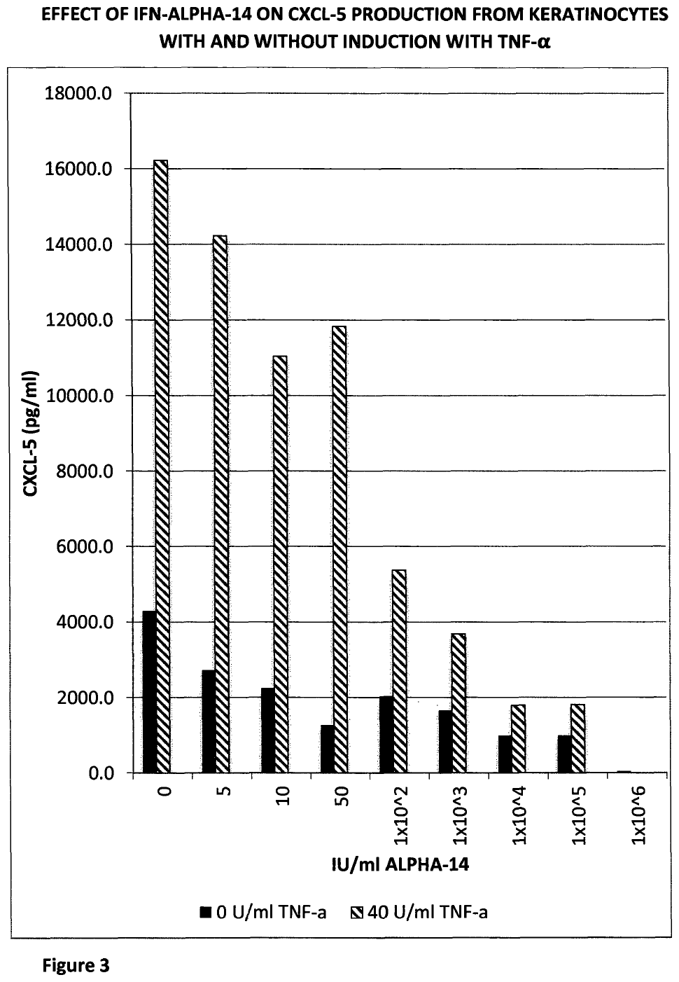

FIG. 3 demonstrates strong inhibition of CXCL5 production. CXCL5 is well known to have chemotactic and activating functions on neutrophils, mainly during acute inflammatory responses. It also maintains neutrophil homeostasis.

Thus all 3 neutrophil chemo-attractants are strongly inhibited by IFNα-14 at low concentrations.

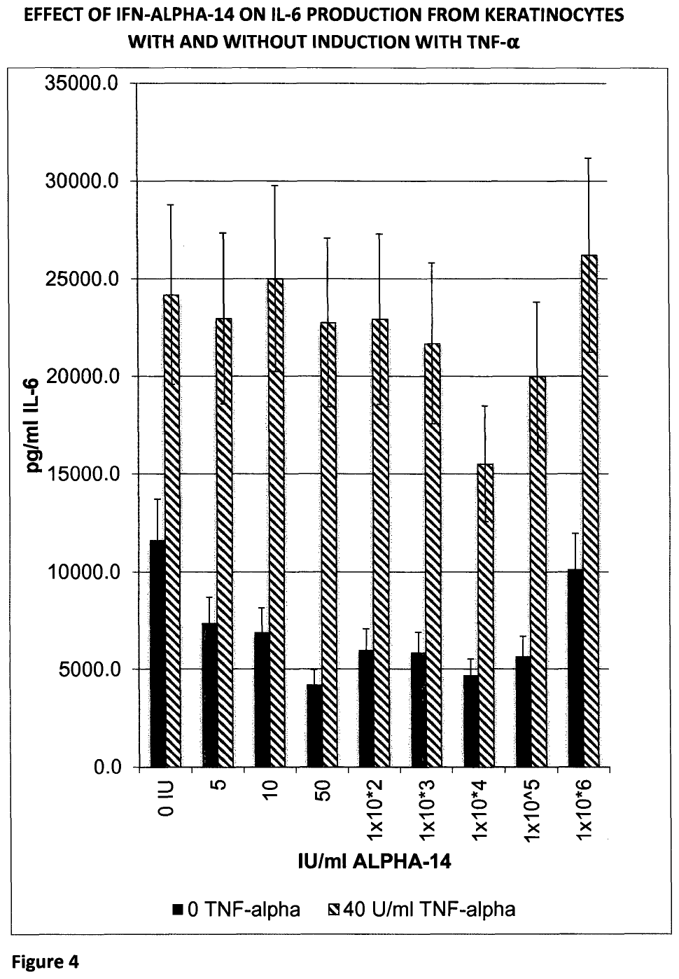

FIG. 4 demonstrates no inhibition of IL6 production in the presence of IFNα-14. Il-6 is a growth facilitator. IFNα-14 has no effect on IL6. This suggests that IFNα-14 will allow skin to continue to grow.

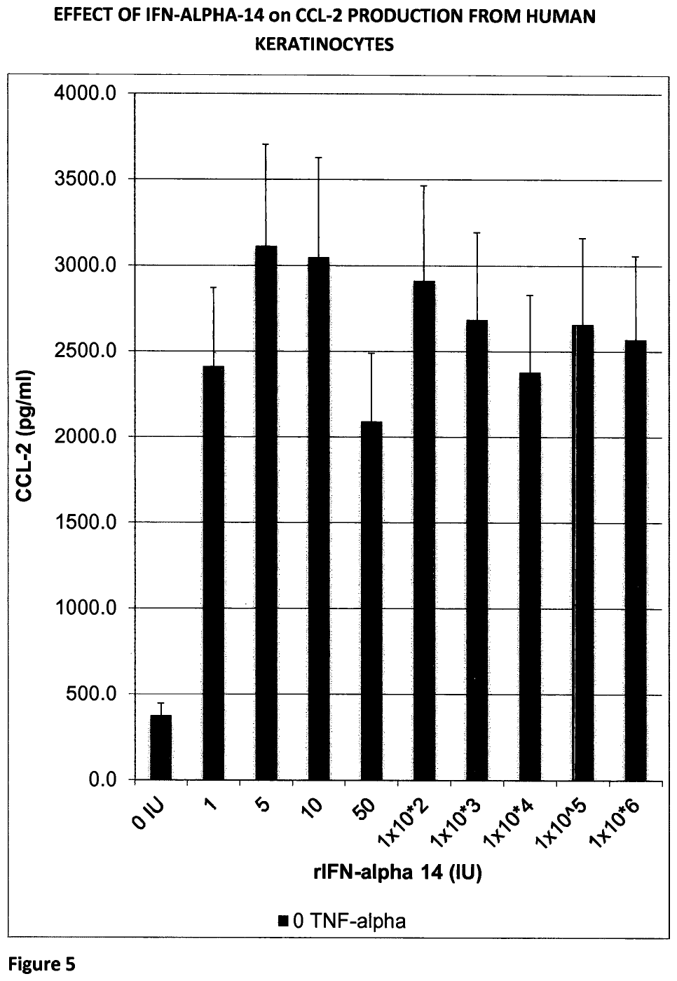

FIG. 5 demonstrates that IFNα-14 induces CCL2. CCL2 is an M2 macrophage chemotactic factor. M2 macrophages are commonly involved in repair of damaged tissues. This is unusual in that it is attracting M2 macrophages that are ‘repair’ cells, to repair keratinocyte damage.

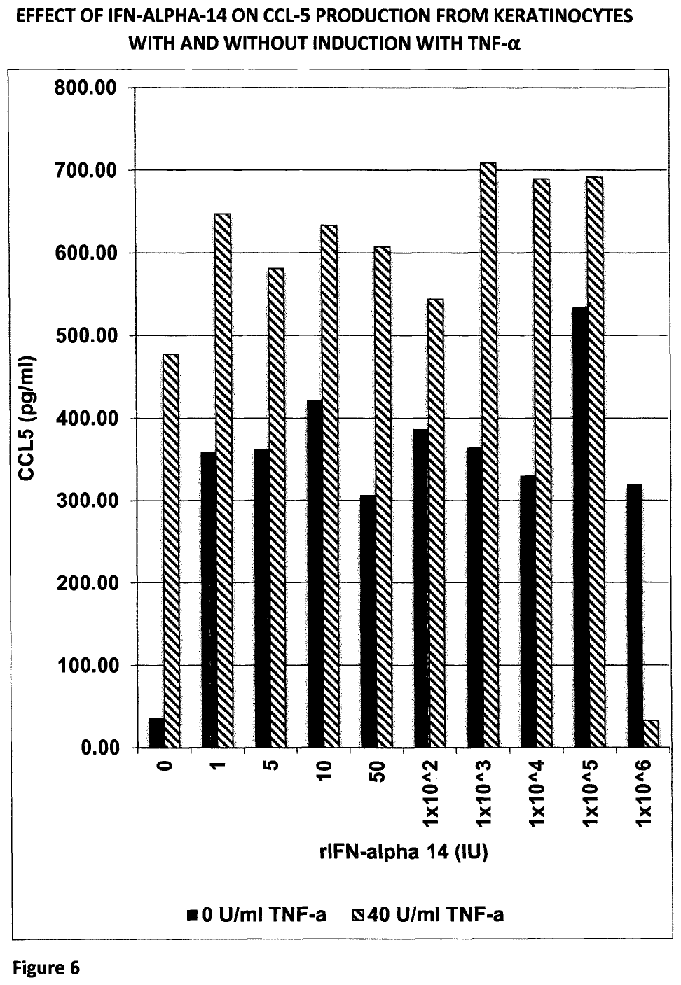

FIG. 6 demonstrates that IFNα-14 induces CCL5. CCL5 is a chemoattractant for T-lymphocytes. This is an indication that IFNα-14 is skewing the response towards a Th1 bias.

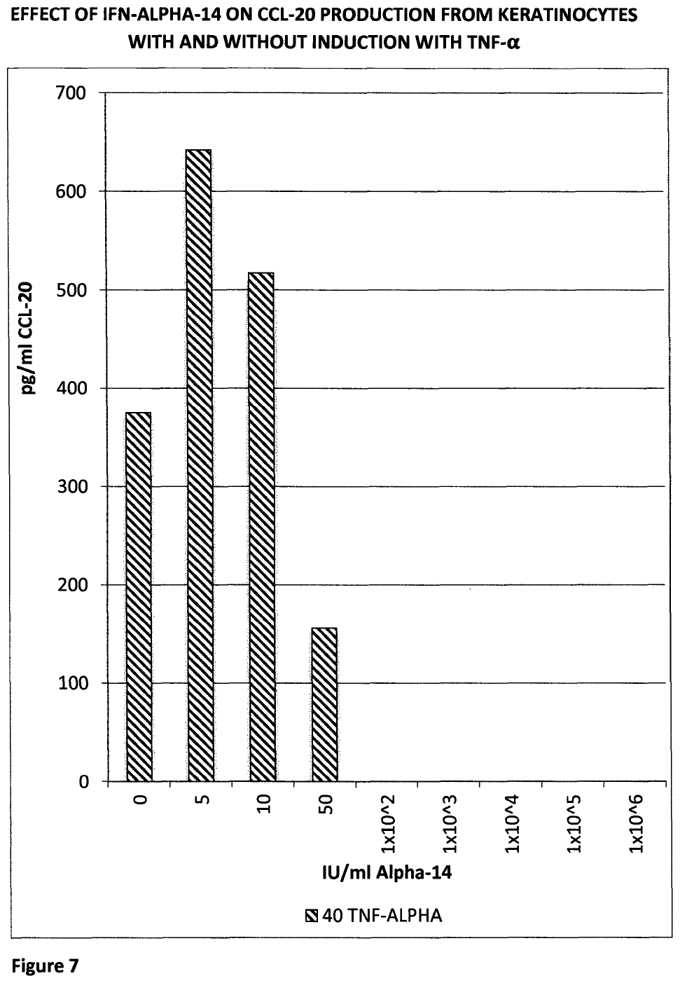

FIG. 7 demonstrates that IFNα-14 inhibits CCL20. CCL20 upregulation represents a danger signal for increased immunosurveillance in barrier disrupted skin and inflammatory skin conditions with impaired barrier function to counteract potential antigen invasion. It attracts lymphocytes and dendritic cells. IFNα-14 stops keratinocytes bringing in CCL20 completely. Such inhibition by IFNα-14 is totally unexpected.

### Experiment 2: The Effect of IFNα-14 on IL17A Production in Skin Biopsies Stimulated into a Psoriatic State

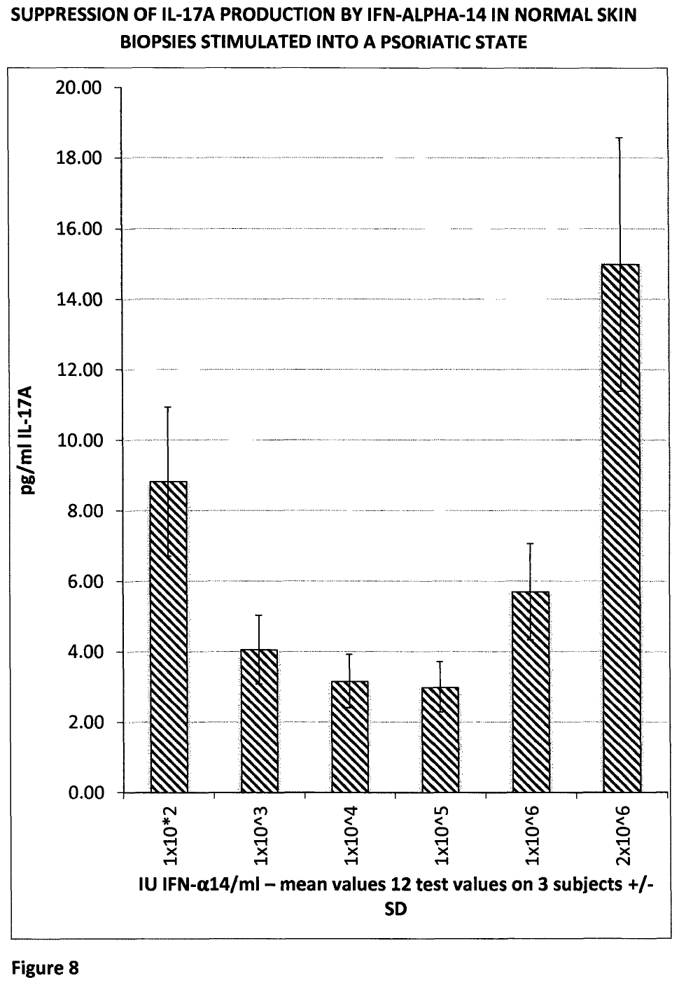

Normal skin biopsies were obtained from healthy subjects and induced into a psoriatic state with a cocktail of cytokines. A biphasic response is well known pharmacologically and is due to the anti-viral properties of IFNα-14. FIG. 8 demonstrates that IL17A was suppressed significantly over a broad range of IFNα-14 concentrations. FIG. 8 supports the hypothesis that IFNα-14 can inhibit the secretion of IL17 in the skin. The desirable therapeutic window for dosing is in the range of 10²-10⁵.

### Experiment 3: The Effect of IFNα-14 on IL17, IL17, IL22, TNF-α, IL6, CXCL8 (IL8) and CXCL1 Production in Whole Human Blood Assays

This experiment uses normal whole human blood. It is stimulated with the lectin PHA (phytohaemagglutinin) to non-specifically activate T lymphocytes. Two doses of PHA are used as there could be a variation in the response (there was not). As this is peripheral blood >96% T lymphocytes are of the alpha-beta receptor type. This is highly indicative of efficacy for systemic treatment only.

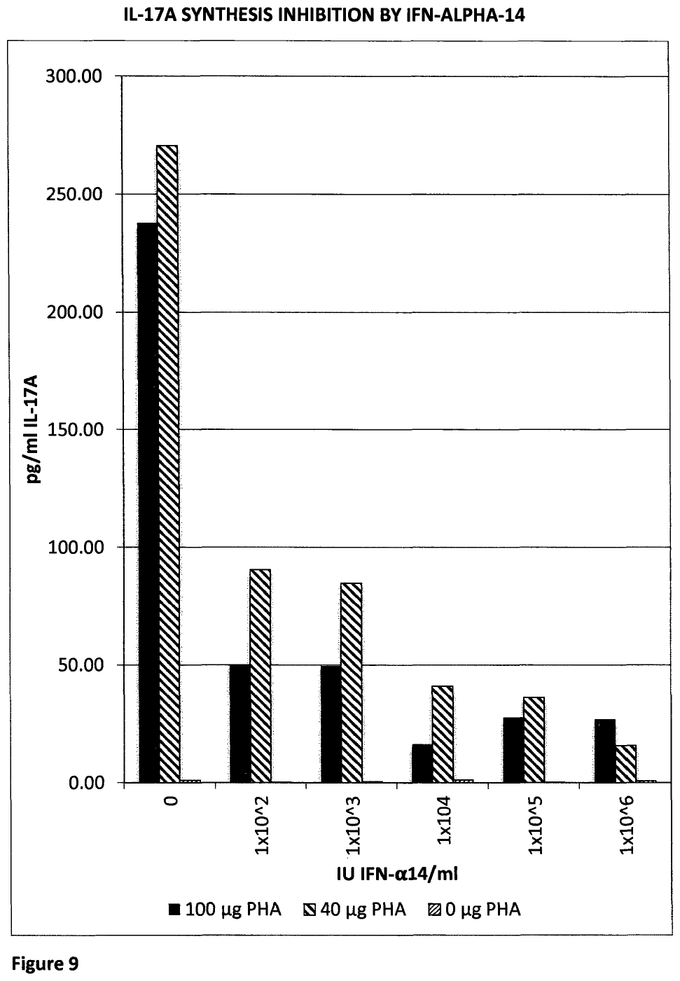

FIG. 9 demonstrates that there was up to an 80% drop in IL17A synthesis from αβ lymphocytes derived from whole human blood with 100 IU/ml IFN-α14 (1 ng/ml). This was an unexpected and remarkable result.

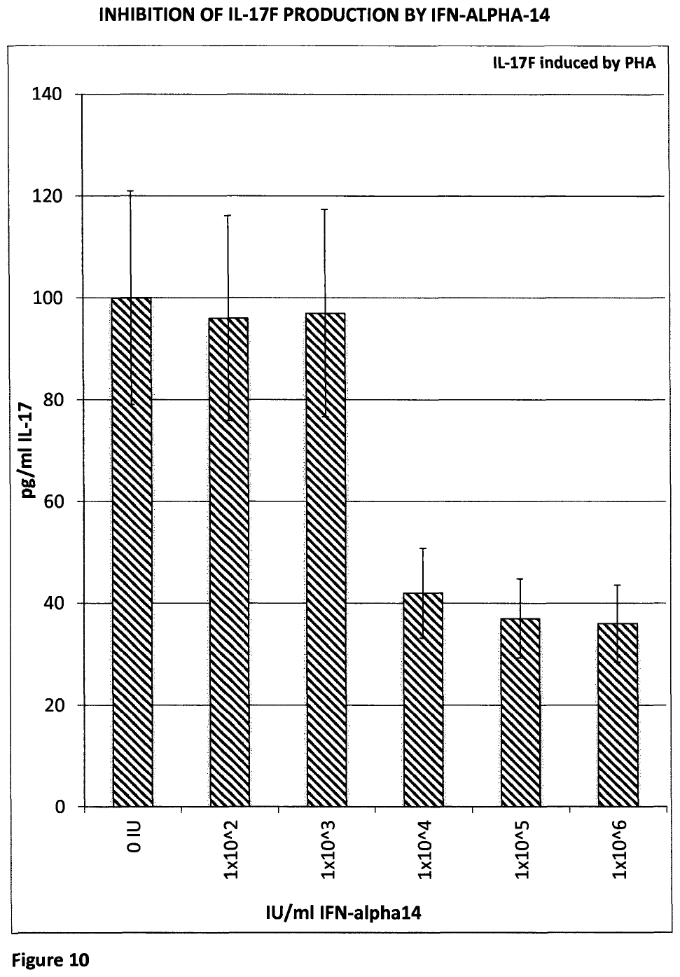

FIG. 10 demonstrates strong inhibition of IL17F synthesis from αβ lymphocytes derived from whole human blood in the presence of IFNα-14 (mean of three subjects). FIG. 10 demonstrates that IFNα-14 significantly inhibits IL17F by up to 87%.

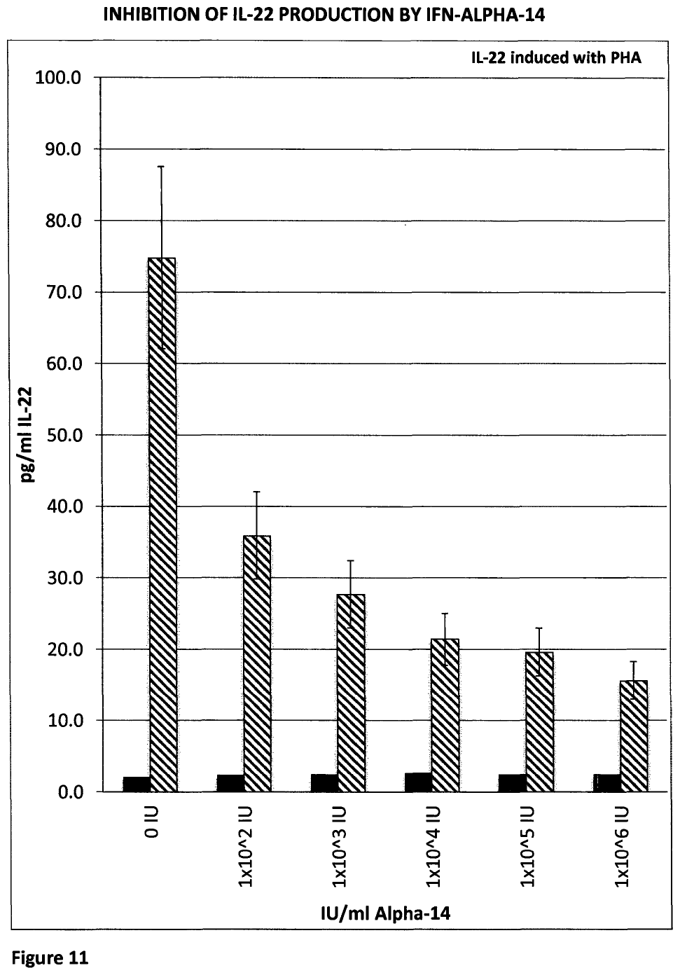

FIG. 11 demonstrates strong inhibition of IL22 production in the presence of IFNα-14. IL22 replicates the chemokine stimulating activity of IL17, hence inhibition of IL22 will also have a positive effect on psoriasis.

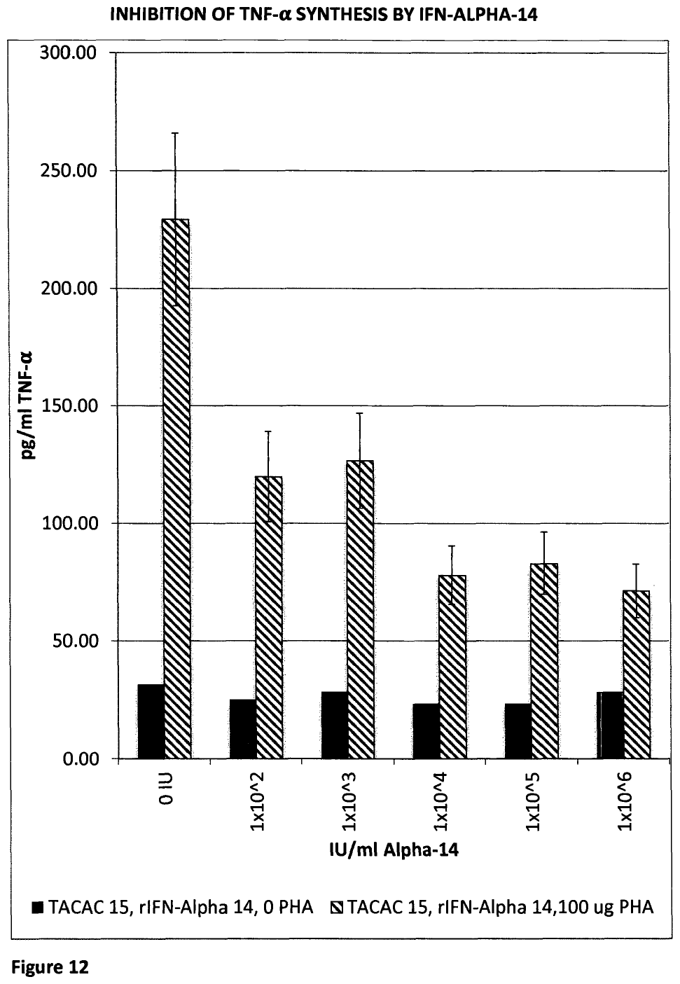

FIG. 12 demonstrates strong inhibition of TNF-α production in the presence of IFNα-14. TNF-α production is induced by 100 g PHA. This 5 day whole blood assay shows >70% reduction in TNF-α production. TNF-α signals endothelial, epithelial cells and keratinocytes to produce chemokines that attract neutrophils. These degranulate to release tissue-damaging chemicals and enzymes that contribute to psoriasis.

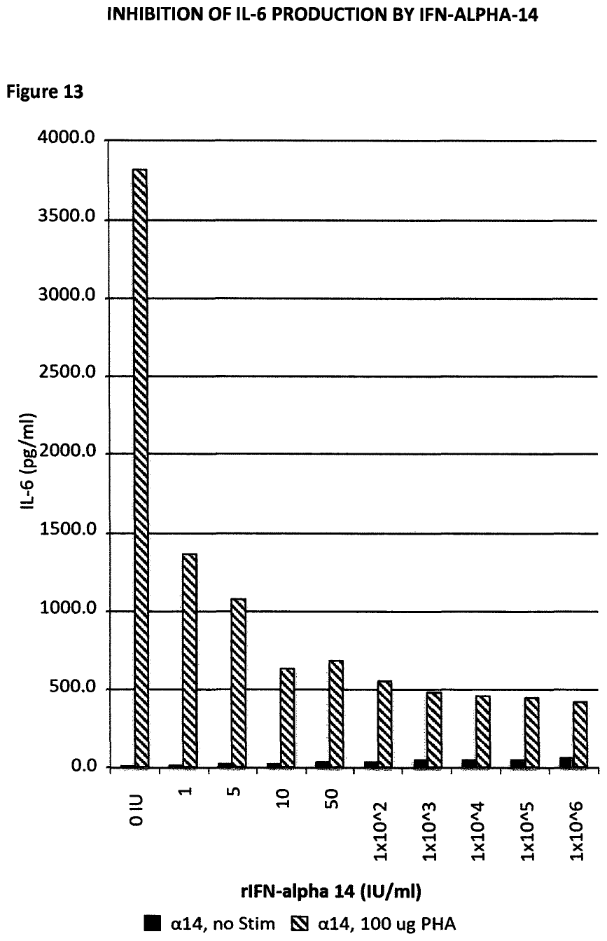

FIG. 13 demonstrates strong inhibition of IL-6 production in the presence of IFNα-14 in a 1 day whole blood assay. IL-6 is an acute phase reactant that rises in trauma and is widely employed as an ancillary growth factor/stimulant. IL6 is a growth factor commonly associated with stress. It is involved in determining the ratio of Tregs to Th17 cells secreting IL17 and thus its removal pushes the T-cell balance away from IL17 secreting Th-17 cells.

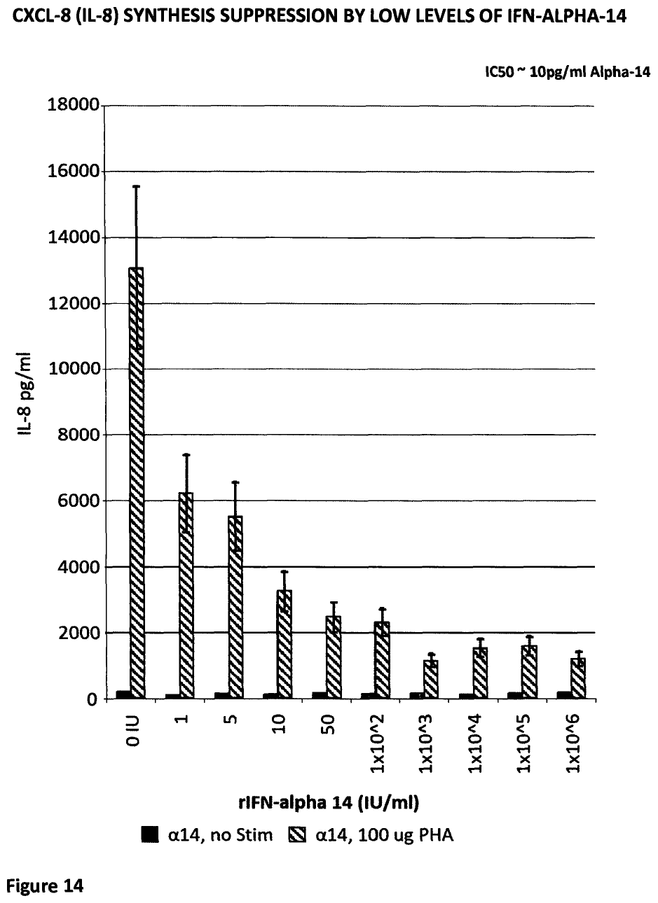

FIG. 14 demonstrates that CXCL8 (IL8) synthesis is inhibited in the presence of very low levels of IFNα-14 in a 1 day whole blood assay. CXCL8 is a major contributor to many aspects of psoriasis and is one of the effector molecules of psoriasis. CXCL8 is a major contributor to inflammation. It attracts neutrophils and basophils/mast cells to the site of inflammation—the latter release histamine and many other noxious agents e.g. prostaglandins, leukotrienes.

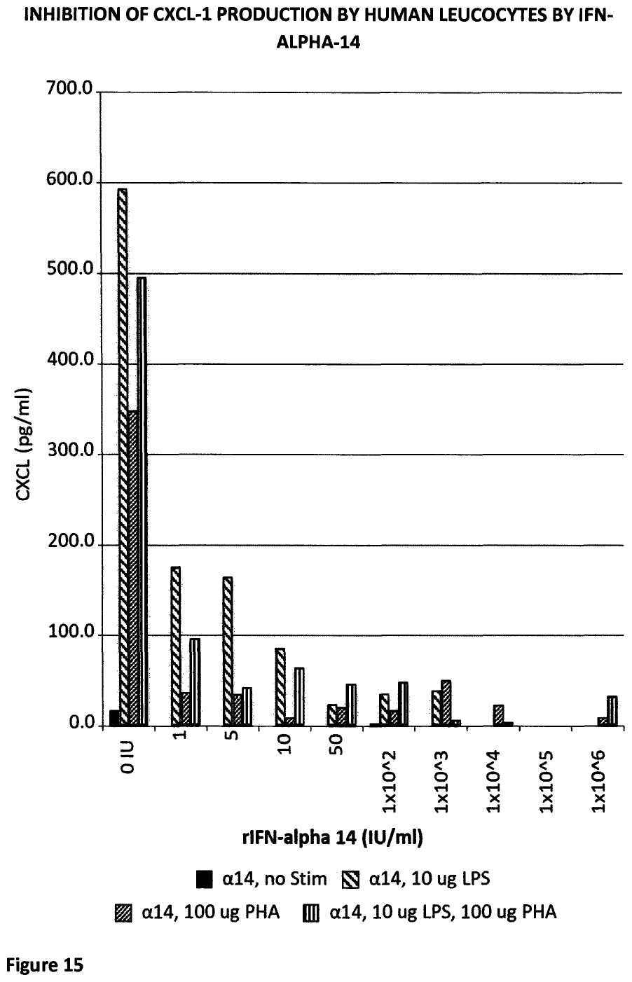

FIG. 15 demonstrates that CXCL-1 production is inhibited in the presence of IFNα-14. CXCL1 is an important keratinocyte chemokine with strong chemotactic characteristics. CXCL1 primarily attracts neutrophils to site of action and is induced by TNF-α. FIG. 8 indicates that stimulation with both LPS or PHA was supressed. The bars indicate stimulation with different levels of LPS or PHA to produce the highest levels possible. This gives IC50s as low as 11 U/ml, showing the potency of IFNα-14. This 1 day CXCL1 whole blood assay indicates how effective the therapy can be.

These results demonstrate that IFNα-14 inhibition of chemokines is a targeted phenomenon. The results indicate that IFNα-14 acts before and after the production of IL17. This is another key differentiator of this therapy compared to other psoriasis therapies.

### Experiment 4: Changes in the Concentrations of 400 Interleukins, Chemokines and CD-Markers in Human Normal Mononuclear Cells, Untreated and Stimulated with 50 Micrograms/Ml Phytohaemagglutinin (PHA) for 3 Days

Human interferon-alpha 10 and 14 were added to cultures at a final concentration of 10s IU/ml and significant changes in concentrations (fold numbers) only are shown. This was carried out with a human BIOMARKER TESTING ARRAY-(RayBiotech Inc.).

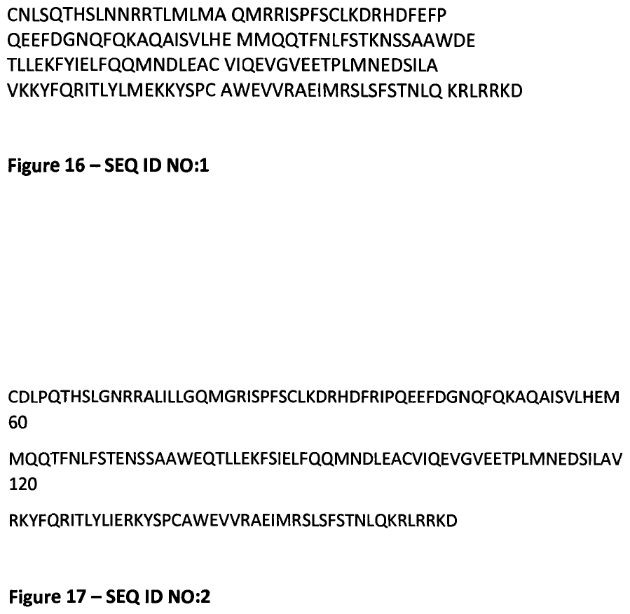

FIG. 16 shows that the α-14 inhibits the synthesis of IL-3 and G-CSF (myeloid cells and granulocytes), IL-5 (eosinophils), IL-13 (Th2 responses and IgE synthesis), as well as IL-17 and 22 (Th17/Th22 cells), as discussed previously. It also enhances IL-12p70 which stimulates alters the immune balance from Th2 towards a more Th1 form. It also inhibits CD-23, the low affinity IgE receptor on basophils/mast cells and suppresses the chemokines, CXCL-1,5 and CCL-1,7,16,20, thus inhibiting any allergic state and associated granulocyte attraction. This strongly suggests a role not only in the control of Psoriasis but also of Atopic Dermatitis.

### Experiment 5: Human IFN-α14 Inhibits Canine IL-17A

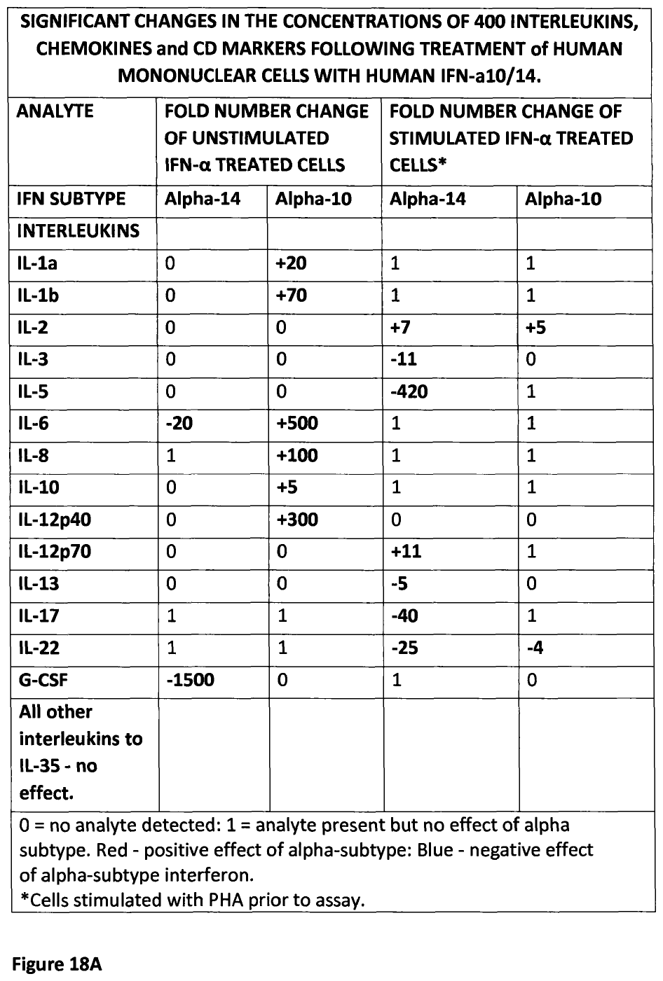

Whole, heparinised blood, was obtained from male Beagle dogs and stimulated with PHA for 3 days in the presence of increasing concentrations of IFN-α14. The results obtained by ELISA are indicated in FIG. 17 and show a marked inhibition of canine IL-17A. This result is very similar to that obtained with human blood (FIG. 9) and human IL-17A. As IL-17A is recognised as a major target for human Psoriasis and Atopic Dermatitis then the canine result is indicative of a response in dogs with the same disorder using human IFN-α14.

### Experiment 6: Treatment of a Dog (Mongrel) with Severe Atopic Dermatitis

A 5 year old male pet dog with severe Dermatitis had been treated with all available medications for this disease. No benefit was seen with any of these therapies and the distressed animal scratched constantly and ate very little—hence its weight was reducing and it was in poor general health.

Human IFN-α14 (104 IU/Kg) in 0.5 ml 0.9% saline was administered sublingually every 2 days over a 2 week period. The animal recovered from its disease state after 3 weeks, was not scratching and was eating normally. Thus this treatment worked where all other therapies used had been unsuccessful. No adverse data was recorded with blood, liver or kidney function tests.

### Experiment 7. Comparison of HYBRID 1 and Interferon-Alpha14 on the PHA-Induced Production of Interleukins/Chemokines from Whole Human Leucocytes

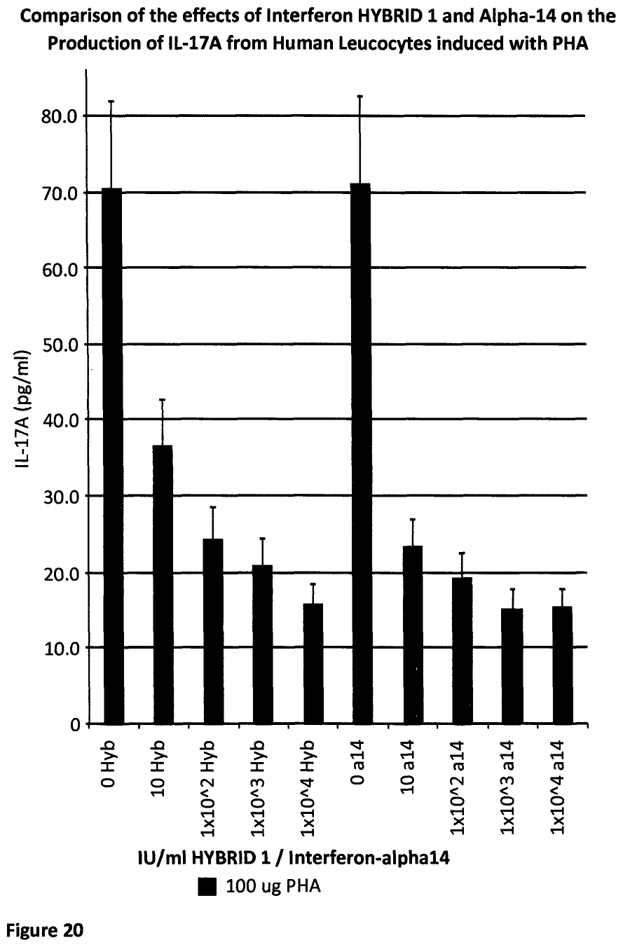

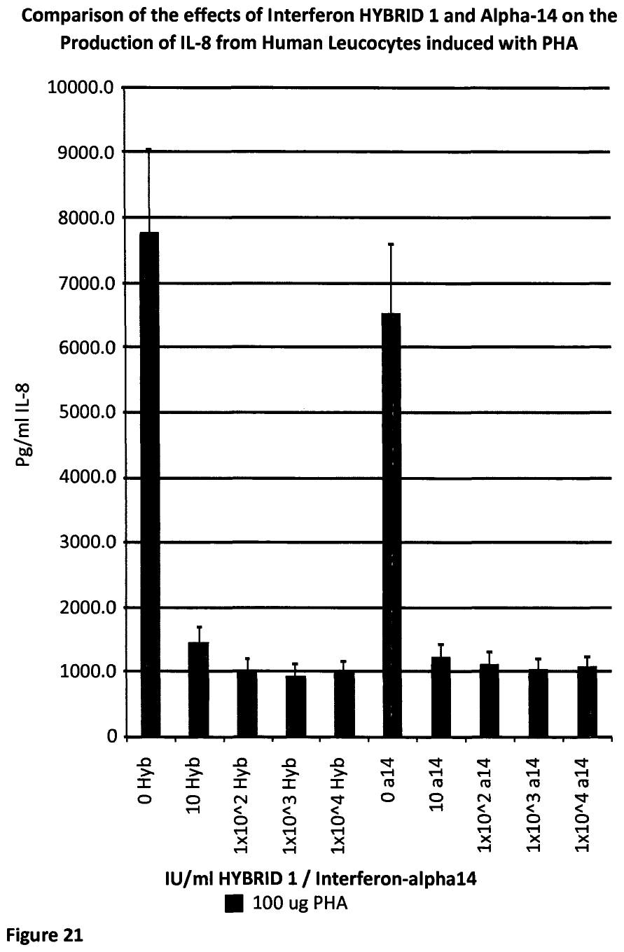

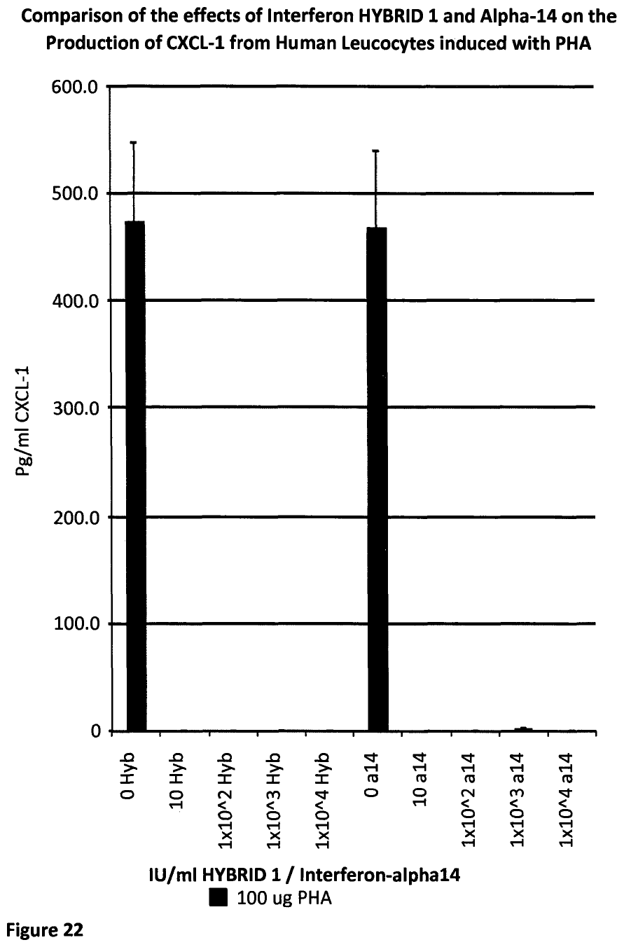

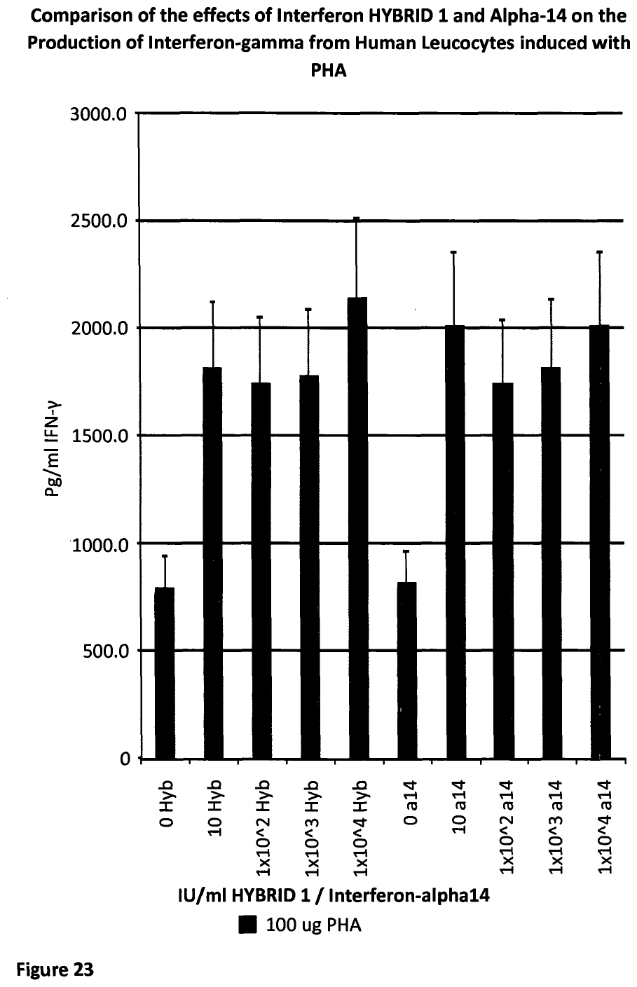

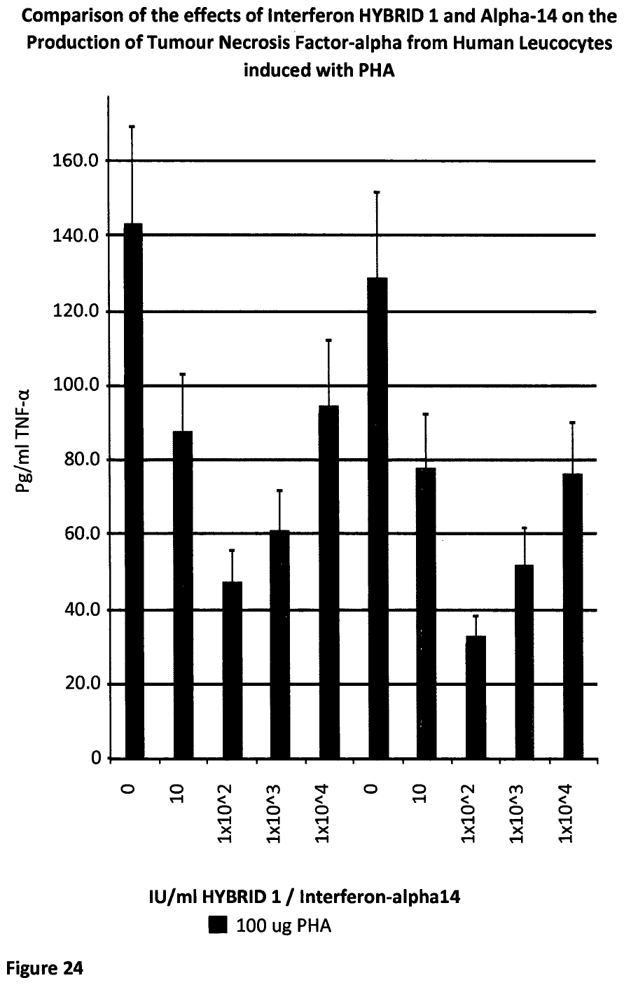

The concentration of the interferons ranged between 0 and 40 IU/ml and the cells where stimulated with 100 micrograms/ml PHA-P for 3 days. The interleukins/chemokines were estimated using commercial ELISAs and IL-17A, IL-8, CXCL-1, TNF-alpha and Interferon-gamma were measured. The results are shown in FIGS. 20 to 24 and no statistical difference was observed at P<0.05 (Students T-test) between the values obtained for Interferon-alpha-14 with those induced by HYBRID1. In essence these 2 molecules where judged to be identical over the 5 assays employed.

Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method for the treatment and/or prophylaxis of psoriasis or atopic dermatitis, said method comprising the step of:
(i) administering to a subject in need thereof a therapeutically effective amount of an interferon alpha subtype, wherein the interferon alpha subtype is IFN-α14, HYBRID 1 or a combination of IFN-α14 and HYBRID 1.

2. The method of claim 1, wherein the interferon alpha subtype IFN-α14 comprises or consists of an amino acid sequence SEQ ID NO:1 or a functionally active fragment or variant thereof.

3. The method of claim 1, wherein the interferon alpha subtype HYBRID 1 comprises or consists of an amino acid sequence SEQ ID NO:2 or a functionally active fragment or variant thereof.

4. The method of any preceding claim, wherein the method of administration is selected from topical administration and sublingual administration.

5. The method of any preceding claim, wherein the therapeutically effective amount of the interferon alpha subtype is a low dose.

6. The method of any preceding claim, wherein the psoriasis is mild, mild-to-moderate, moderate, moderate-to-severe or severe psoriasis.

7. An interferon alpha subtype, wherein the interferon alpha subtype is IFN-α14, HYBRID 1 or a combination of IFN-α14 and HYBRID 1 for use in the treatment and/or prophylaxis of psoriasis or atopic dermatitis.

8. The interferon alpha subtype of claim 7, wherein the IFN-α14 comprises or consists of an amino acid sequence SEQ ID NO:1 or a functionally active fragment or variant thereof.

9. The interferon alpha subtype of claim 7, wherein the interferon alpha subtype HYBRID 1 comprises or consists of an amino acid sequence SEQ ID NO:2 or a functionally active fragment or variant thereof.

10. The interferon alpha subtype of claims 7 to 9, wherein the interferon alpha subtype is administered topically or is by sublingual administration.

11. The interferon subtype of claim 10, wherein the interferon alpha subtype is administered at a low dose.

12. The interferon alpha subtype of claims 7-11 wherein, the psoriasis is mild, mild-to-moderate, moderate, moderate-to-severe or severe psoriasis.

13. A composition comprising an interferon alpha subtype, wherein the interferon alpha subtype is IFN-α14, HYBRID 1 or a combination of IFN-α14 and HYBRID 1, for use in the treatment and/or prophylaxis of psoriasis or atopic dermatitis.

14. A pharmaceutical composition comprising an interferon alpha subtype, wherein the interferon alpha subtype is IFN-α14, HYBRID 1 or a combination of IFN-α14 and HYBRID 1, for use in the treatment and/or prophylaxis of psoriasis or atopic dermatitis.
